(19) United States
(12) Patent Application Publication
(10) Pub. No.: US 2024/0167962 A1
(43) Pub. Date: May 23, 2024

(54) SYSTEM AND METHOD FOR AUTOMATIC MONITORING OF PAVEMENT CONDITION

(71) Applicant: IRIS R&D Group Inc., Burlington (CA)

(72) Inventors: Emil Sylvester Ramos, Burlington (CA); David Keaney, Oakville (CA); David Moody, Stoney Creek (CA)

(21) Appl. No.: 18/281,723

(22) PCT Filed: Mar. 15, 2022
(86) PCT No.: PCT/CA2022/050391

§ 371 Date: Sep. 12, 2023

Related U.S. Application Data
(60) Provisional application No. 63/160,983, filed on Mar. 15, 2021.

(51) Int. Cl. G01N 21/88; G01P 15/14; G01S 17/89; G01S 17/931; G06T 7/00; G06T 7/73

(52) U.S. Cl. CPC G01N 21/88; G01S 17/89; G01S 17/931; G06T 7/0004; G06T 7/75; G01N 2021/8864; G01N 2021/888; G01N 2201/0216; G01P 15/14; G06T 2207/30252

(57) ABSTRACT

Various embodiments are provided herein for a system and method for automatic monitoring of pavement condition. In at least some embodiments provided herein, there is provided a method for assessing a condition of a pavement segment, the method comprising: analyzing, using a trained machine learning model, an image of the pavement segment; generating, by the machine learning model, an output comprising one or more identified first pavement distresses in the pavement segment; analyzing accelerometer data associated with the pavement segment; based on the analyzing, determining one or more identified second pavement distresses in the pavement segment; and based on the one or more identified first and second pavement defects, determining pavement condition data associated with the pavement segment.

### FIELD

The described embodiments relate to monitoring of pavement condition, and in particular, to a method and system for automatic monitoring of pavement condition.

### INTRODUCTION

Distresses, such as cracks, potholes and discontinuities, often develop in asphalt pavement owing to factors that include, inter alia, heavy traffic load, changing environment and weather-related conditions, drainage problems as well as road construction deficiencies. At minimum, the presence of minor pavement distresses (e.g., bumps and corrugations) on pavement can have the effect of deteriorating the overall ride experience for passengers of vehicles driving over the pavement. In many cases, however—where left untreated—minor pavement distress can develop into more significant pavement defects (e.g., potholes), which may become more critical motorway hazards.

To ensure road networks are up to safety and comfort standards, regional authorities often enact regulations that setout minimum maintenance standards for the quality of paved roads. Compliance with these standards is observed by deploying teams of road inspectors who visually inspect and assess pavement condition. The condition of various pavement sections may be reported using one of a number of established measures, including most notably, the pavement condition index (PCI) and/or the international roughness index (IRI).

### SUMMARY OF VARIOUS EMBODIMENTS

In at least one broad aspect described herein, there is provided a method for assessing a condition of a pavement segment, the method comprising: analyzing, using a trained machine learning model, an image of the pavement segment; generating, by the machine learning model, an output comprising one or more identified first pavement distresses in the pavement segment; analyzing accelerometer data associated with the pavement segment; based on the analyzing, determining one or more identified second pavement distresses in the pavement segment; and based on the one or more identified first and second pavement defects, determining pavement condition data associated with the pavement segment.

In at least one embodiment, the image and accelerometer data are captured by a camera and accelerometer of an edge device.

In at least one embodiment, the edge device is mounted to a vehicle.

In at least one embodiment, the method further comprises analyzing the pavement condition data to determine one or more pavement condition outputs, associated with the pavement segment.

In at least one embodiment, the pavement condition output corresponds to a pavement condition index (PCI) value.

In at least one embodiment, analyzing the image using the trained machine learning model comprises: applying feature extraction layers to the image to generate a feature map; generating a set of anchor boxes in the image to generate a plurality of anchor boxes; inputting the plurality of anchor boxes and the feature map into a regional proposal network (RPN) layer to identify the locations of the pavement defects in the image.

In at least one embodiment, the feature extraction layer comprises a ResNet-50 model.

In at least one embodiment, the method further comprises the trained machine learning model classifying the pavement defect types.

In at least one embodiment, classifying the pavement defect types is generated by feeding the feature extraction map and proposal generated by the RPN layer, into an ROI (region of interest) pooling layer and a regressor.

In at least one embodiment, prior to applying the trained machine learning, applying a tilt correction to the input image.

In another broad aspect described herein, there is provide a system for assessing a condition of a pavement segment, the system comprising: an edge device comprising an image sensor for capturing an image of the pavement segment, and at least one of a motion and orientation sensor for recording respective motion and orientation data associated with the pavement segment; and a server comprising a processor configured to: receive, from the edge device, the image and at least one of motion and orientation data; analyze, using a trained machine learning model, the image of the pavement segment; generate, by the machine learning model, an output comprising one or more identified first pavement distresses in the pavement segment; analyze at least one of the motion and orientation data associated with the pavement segment; based on the analyzing, determine one or more identified second pavement distresses in the pavement segment; and based on the one or more identified first and second pavement defects, determine a pavement condition output associated with the pavement segment.

In at least one embodiment, the edge device is mounted to a vehicle.

In at least one embodiment, the motion data comprises accelerometer data.

In at least one embodiment, the processor is further configured to analyze the pavement condition data to determine one or more pavement condition outputs, associated with the pavement segment.

In at least one embodiment, the pavement condition output corresponds to a pavement condition index (PCI) value.

In at least one embodiment, analyzing the image using the trained machine learning model comprises the processor being further configured to: apply feature extraction layers to the image to generate a feature map; generate a set of anchor boxes in the image to generate a plurality of anchor boxes; input the plurality of anchor boxes and the feature map into a regional proposal network (RPN) layer to identify the locations of the pavement defects in the image.

In at least one embodiment, the feature extraction layer comprises a ResNet-50 model.

In at least one embodiment, the trained machine learning model classifying the pavement defect types.

In at least one embodiment, classifying the pavement defect types is generated by feeding the feature extraction map and proposal generated by the RPN layer, into an ROI (region of interest) pooling layer and a regressor.

In at least one embodiment, prior to applying the trained machine learning, applying a tilt correction to the input image.

In other embodiments described herein, there is provided a system for assessing a condition of a pavement segment, the system comprising: an edge device comprising an image sensor for capturing an image of the pavement segment, and at least one of a motion sensor and an orientation sensor for recording respective motion and orientation data associated with the pavement segment; and a server comprising a processor configured to: receive, from the edge device, the image and sensor data; analyze, using a first trained machine learning model, the image of the pavement segment; generate, by the first machine learning model, a first output comprising one or more identified first pavement distresses in the pavement segment; analyze, using a second trained machine learning model, at least one of the motion and orientation data associated with the pavement segment; generate, by the second machine learning model, a second output comprising one or more identified second pavement distresses in the pavement segment; and based on the one or more identified first and second pavement defects, determine a pavement condition output associated with the pavement segment.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A is an illustrative example of a road segment having one or more pavement distresses;

FIG. 1B is an illustration of an example method of determining a pavement condition index (PCI) for a pavement segment;

FIG. 1C is an example method for determining the PCI value of an asphalt concrete pavement segment in accordance with established standards;

FIG. 1D is an example deduct value plot for a corrugation distress;

FIG. 2A is an illustration of an example embodiment of an environment in which the methods and systems disclosed herein may operate;

FIG. 2B is a simplified block diagram for an example embodiment of a system for automated monitoring of pavement condition, in accordance with some embodiments;

FIG. 3A is a simplified block diagram of an example embodiment of an edge device, in accordance with some embodiments;

FIG. 3B is a simplified block diagram of an example embodiment of a server, in accordance with some embodiments;

FIG. 4 is an example embodiment of a method for automatic monitoring of pavement condition, in accordance with some embodiments;

FIG. 5A is an example road network map showing pavement segments of a road network which are color coded according to their determined PCI value;

FIG. 5B is an example road network map showing pavement segments of a road network labeled with the locations of various detected pavement distresses;

FIG. 6 is an example embodiment of a method for automatic monitoring of pavement condition of a pavement segment, in accordance with some other embodiments;

FIG. 7A is an example embodiment of a method for using a trained machine learning model to process image data to determine the pavement condition of a pavement segment;

FIG. 7B is an example embodiment of a method for training an image analysis machine learning model to analyze images of pavement segments to determine pavement condition, in accordance with some embodiments;

FIG. 8A is an example labelled image frame showing one or more pavement distresses on a pavement segments;

FIG. 8B is an example labeled image showing the location of a pavement distress on a pavement segment as generated by a trained machine learning model;

FIG. 8C is an example distorted image and including an object with known dimensions;

FIG. 8D is an example output file that includes pavement distress data for a pavement segment;

FIG. 9A is an example embodiment of a method for processing motion and/or orientation data to identify ride-based pavement distresses in a pavement segment, according to some embodiments;

FIG. 9B is an example embodiment of a method for processing motion and orientation data to identify ride-based pavement distresses in a pavement segment, according to some other embodiments;

FIG. 9C is an example embodiment of a method for training a sensor data machine learning model to analyze sensor data of pavement segments, in accordance with some embodiments;

FIG. 10A is an example plot of vertical acceleration data collected in association with one or more example ride-based pavement distresses;

FIG. 10B is another example plot of vertical acceleration data collected in association with one or more example ride-based pavement distresses;

FIG. 100 is still another example plot of vertical acceleration data collected in association with one or more example ride-based pavement distresses;

FIG. 11 is an example embodiment of a method for determining pavement condition outputs for a pavement segment based on one or more of image and sensor data;

FIG. 12 is an example embodiment of a method for determining pavement condition, in accordance with other embodiments;

FIGS. 13A-13D are example architectures for machine learning models used for analyzing images for pavement defects; and

FIG. 13E is an example schematic of an image of a pavement segment.

Further aspects and features of the example embodiments described herein will appear from the following description taken together with the accompanying drawings.

### DESCRIPTION OF VARIOUS EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the example embodiments described herein.

It should also be noted that the terms “coupled” or “coupling” as used herein can have several different meanings depending in the context in which these terms are used. For example, the terms coupled or coupling can have a mechanical, chemical or electrical connotation. For example, as used herein, the terms coupled or coupling can indicate that two elements or devices can be directly connected to one another or connected to one another through one or more intermediate elements or devices via an electrical or magnetic signal, electrical connection, an electrical element or a mechanical element depending on the particular context. Furthermore coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

The headings and Abstract of the Disclosure provided herein are for convenience only and do not interpret the scope or meaning of the embodiments.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Examples of communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Examples of communicative couplings include, but are not limited to, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

At least a portion of the example embodiments of the apparatuses or methods described in accordance with the teachings herein may be implemented as a combination of hardware or software. For example, a portion of the embodiments described herein may be implemented, at least in part, by using one or more computer programs, executing on one or more programmable devices comprising at least one processing element, and at least one data storage element (including volatile and non-volatile memory). In some cases, these devices may also have at least one input device (e.g., a touchscreen, and the like) and at least one output device (e.g., a display screen, a printer, a wireless radio, and the like) depending on the nature of the device, such as the controller 1700, for example.

It should also be noted that there may be some elements that are used to implement at least part of the embodiments described herein that may be implemented via software that is written in a high-level procedural language such as object-oriented programming. The program code may be written in JAVA, C, C⁺⁺ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media (e.g., a computer readable medium such as, but not limited to, ROM, flash memory, magnetic disk, optical disc) or a device that is readable by a programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, DVD, tapes, chips, and magnetic, optical and electronic storage. In at least one alternative embodiment, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

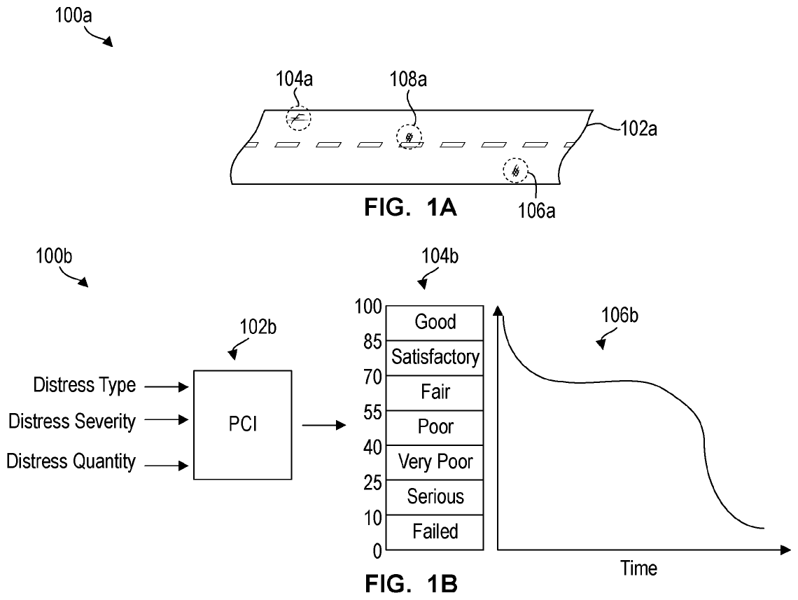

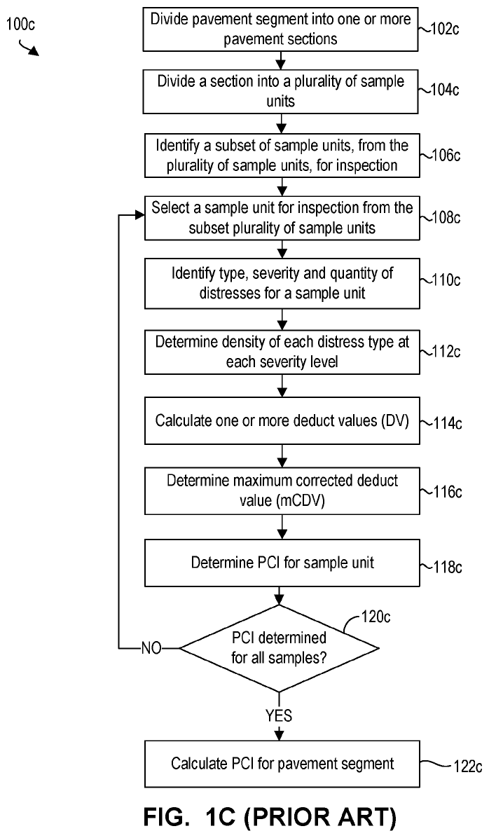

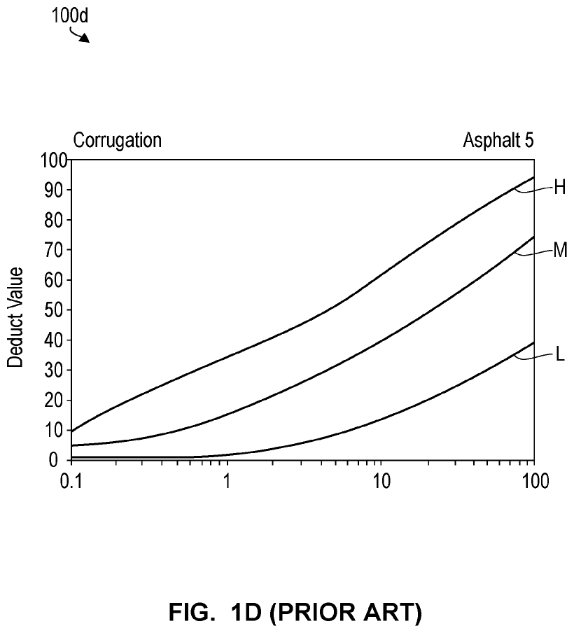

As explained in the background discussion, pavement distresses often develop in road networks, as well as in other pavement covered areas (e.g., parking lots), owing to various factors that include, inter alia, heavy traffic load, weather and environmental conditions, poor road construction and drainage deficiencies. FIG. 1A, for example, illustrates example pavement distresses that can develop in different locations along a pavement segment 102a, such as along the edges of the pavement (e.g., pavement distresses 104a, 106a) and/or along the center-line of the road (e.g., pavement distress 108a).

Regional authorities, tasked with monitoring road infrastructure, often rely on well-established standardized measures for assessing and reporting pavement condition. In particular, these measures assist authorities, and other entities, in making objective assessments of when pavement maintenance is required, or otherwise, when pavement condition is below the minimum regulated maintenance standards. One known example measure for expressing pavement condition is called the pavement condition index (PCI), which is a numerical index that expresses the structural integrity, surface operation condition and ride comfort of a given pavement segment. Other example measures for evaluating pavement condition also include the international roughness index (IRI), which quantifies pavement irregularities that adversely affect ride quality of a vehicle, as well as the present serviceability rating (PSR). Still other types of standards include various regional standards, such as the SPO-24 Pavement Surface Condition rating, developed by the Ministry of Transportation of Ontario, Canada.

More particularly, the method and process of determining the PCI for a pavement segment has been standardized by the American Society for Testing Materials (ASTM), in a standard known as the ASTM D6433-11 standard. According to the ASTM D6433-11 standard, eighteen categories of pavement distresses exist, including alligator cracks, bleeding, block cracking, bumps and sags, corrugations, depressions, edge cracking, joint reflections, lane/shoulder drop-offs, longitudinal and transverse cracking, patching and utility cut patching, polished aggregate, pot holes, rutting, shoving, slippage cracking, swelling and weathering and raveling.

To determine the PCI of a road segment using the ASTM D6433-11 standard, inspectors visually survey pavement surface condition in what is often termed as a “windshield inspection”. To this end, the inspectors carry a data sheet and, based on visual observation, manually record: (a) the different types of observed distresses, (b) the severity level of each of these distresses, and (c) the size or length of the observed distresses. In some cases, the inspectors may also carry a hand odometer wheel to assist in measuring the distress size.

In order to assist inspectors in classifying the type and severity of observed distresses, the ASTM D6433-11 standard also includes a detailed textual explanation of each type of distress, as well as a three-tier rating system for classifying the severity of each distress (e.g., low severity, medium severity and high severity). In various cases, the ASTM standard also expresses the severities of various distress in visual terms (e.g., small cracks versus a highly developed network of pattern of cracks). However, for at least five of the distress types (e.g., bumps, corrugations, railroad crossing, shoving and swells), the severity is not determined visually, but is determined based on ride quality metrics. In particular, to assess severity of ride quality distresses, the inspector is expected to drive a vehicle over the distress, e.g., at normal operating speed, and gauge the distress severity based on the degree of vehicle vibration.

Referring to FIG. 1B, as shown, the PCI for a pavement segment is determined by combining collected data in respect of identified distress types, as well as the severity level of each distress type, and the quantity of each identified distress type at each level of severity. The PCI calculation 102b then yields a numerical index value 104b which is in a range between ‘0’ (i.e., failed) and ‘100’ (i.e., ‘good’), indicative of pavement condition. If the pavement condition is below a “fair” rating (i.e., 55 to 70), repairs may be required to the pavement section. The example plot 106b illustrates the general PCI trajectory for a pavement section over the course of time.

Reference is now made to FIG. 1C, which shows a simplified method 100c for determining the PCI value of an asphalt concrete pavement segment in accordance with the ASTM 6433 standard.

As shown, at 102c, a pavement segment that is being analyzed for its PCI is divided into one or more pavement sections. At 104c, each pavement section is further divided into a plurality of sample units. At 106c, a subset of the plurality of the sample units is selected for inspection. The ASTM 6433 standard provides one or more mathematical formulas for determining a minimum number of sample units to inspect in order to achieve a desired confidence level.

At 108c, a given sample unit, of the subset plurality of sample units determined at act 106c, is selected for inspection. At 110c, the selected sample unit is visually inspected (e.g., by an inspector) to identify the type of pavement distresses located in the sample unit, the severity of each type of identified pavement distresses (i.e., high, medium or low), and the size of each identified pavement distress at each level of severity (e.g., a measure of the size, length or count number, etc.).

At 112c, the density of each distress type at each severity level is determined. For example, this is determined in accordance with equation (1):

$$Density = \frac{Cumulative Quantity of Distress Types at a Severity Level}{Area of Sample Unit} \qquad ( 1 )$$

In particular, the density for each distress type at a given severity level is determined by dividing the cumulative quantity of all distress types at the given severity level by the sample unit area. The cumulative quantity of the distress type at a severity level is determined by aggregating, the size, length or count number for each observed distress type at a given severity level as stipulated by the standard.

At 114c, for each distress type at each severity level, a deduct value (DV) is calculated. In particular, the deduct values are calculated using one or more non-linear deduct value curves available in the ASTM standard. For example, FIG. 1D shows an example deduct value (DV) plot for corrugations in asphalt, and which relates different distress densities (in percentage, between 0.1 to 100%), at different corrugations severity levels (e.g., low, medium and high severities), to different pre-determined deduct values.

At 116c, the deduct values (DVs), determined for each distress type and severity level, are organized in descending order (i.e., highest DV to lowest DV). An allowable number of DVs is then determined using one or more known equations. For example, the allowable number of DVs can correspond to the ten highest DV values in the ordered descending arrangement. The allowable DVs are then summed to determine a total deduct value (TDV). A value “q” is further determined and may correspond, for example, to the number of allowable DVs having a value greater than a pre-determined threshold (e.g., 2.0). A maximum corrected deduct value (mCDV) is calculated by using one or more polynomial function parameters that determine the CDV based on the TDV and the “q” value.

At 118c, the PCI for the sample unit is determined based on the equation PCI=100-mCDV. At 120c, it is determined whether the PCI has been determined for all sample units in the pavement segment. If not, the method 100c can return to 108c to determine the PCI for the next sample unit. Otherwise, the PCI for the pavement segment is determined through a weighted average calculation of the PCI values for each inspected subsample unit.

As stated earlier, the standard method of assessing pavement condition (e.g., PCI)—based on visual surveys conducted by inspectors deployed to assess road networks—is often fraught with difficulties.

For example, significant cost, time and manpower is often required to conduct these visual surveys, especially for expansive road networks. As a result, regional authorities with limited budgets often struggle to balance pavement inspection expenses, with other maintenance expenses (e.g., snow clearances, etc.). For this reason, it is not uncommon for regional authorities to limit road pavement surveys to occur only annually, or even bi-annually. As a result, many road networks are underkept, which can result in hazardous driving conditions. In many cases, authorities resort to relying on public reports of pavement problems in order to maintain road networks.

Additionally, the conventional method of visually surveying pavement condition may be highly subjective, as well as error-prone. In particular, inspectors may not always correctly visually classify pavement distresses, as well as classify the severity of these distresses (i.e., in accordance with the established standards). In turn, this can result in erroneous or inaccurate pavement condition reports. These errors often compound for surveys involving large networks of roads with many interspersed defects of varying severities. In order to reduce the incidences of error, it is often necessary to deploy highly specialized inspectors to conduct the visual surveys.

Still further, the process of deploying inspectors to road network may incur direct risk hazards for these inspectors. For example, inspectors operating in high traffic zones may be at risk of fatal collision with vehicles sharing the roadway.

In recent years, to ameliorate at least some of the above-noted challenges, there have been some attempts to deploy specialized vehicles (e.g., vans and trucks) that are equipped with large scanning hardware, such as ground penetrating radar (GPR) systems. These vehicles, which are used to at least partially replace human inspectors, rely on large scanning devices to collect below surface pavement condition data to determine pavement condition. However, these solutions also often suffer from significant drawbacks, including very high expenses for purchasing the scanning hardware, as well as the requirement for specially fitted vehicles that are equipped to carry the large sized scanning hardware. Accordingly, the use of these systems is often highly inconvenient, and regional budgets can only accommodate purchasing a limited number of these specially fitted vehicles.

In view of the foregoing, there is a desire for a method and system that allows for automated monitoring of pavement condition. In particular, there is a desire for a method and system which can automatically monitor pavement condition in real-time or near real-time. There is also desire for a method and system that provides automated monitoring of pavement condition in a simplified, cost-effective and easily deployable manner.

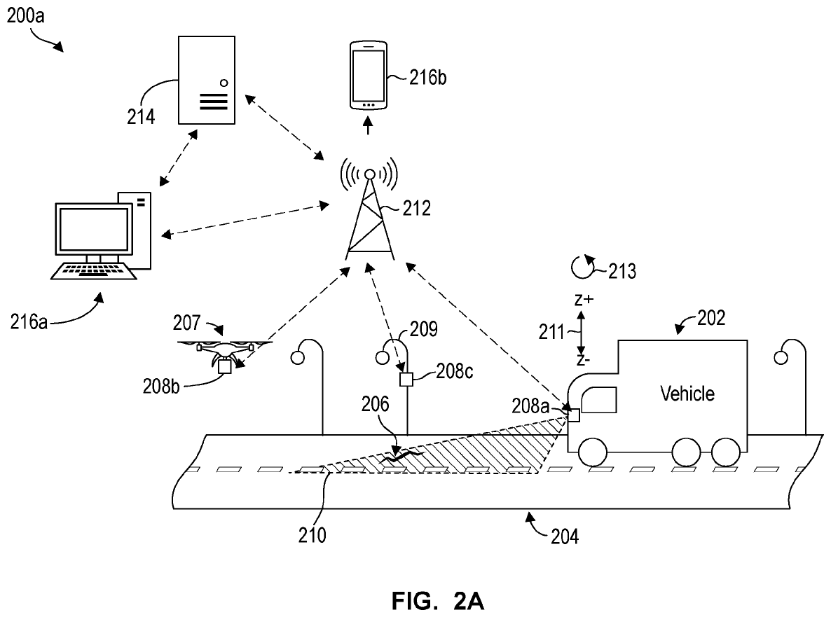

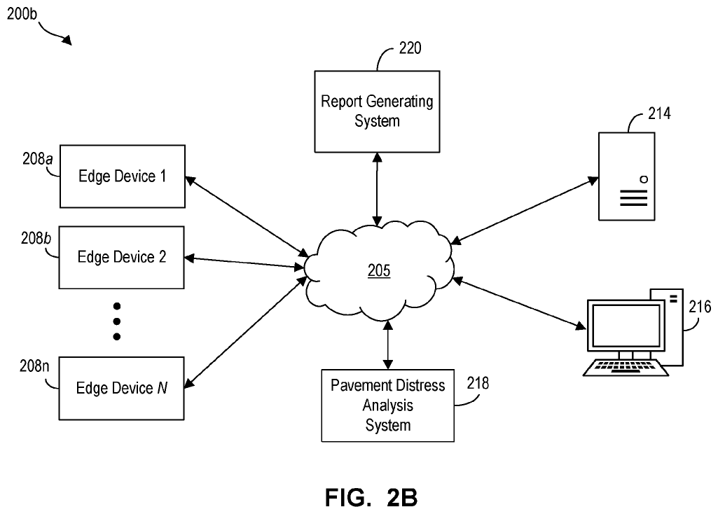

Referring now to FIG. 2A, which illustrates an example embodiment of an environment 200a in which the methods and systems disclosed herein may operate.

As shown, and in various embodiments disclosed herein, automated monitoring of pavement condition may occur using one or more edge devices 208. The edge devices 208 may be installed (i.e., mounted) to various stationary or moving structures. For instance, in the illustrated example, an edge device 208a may be mounted on a vehicle 202 traversing a road segment 204. The edge device 208a can be mounted, for example, to the vehicle windshield such that the edge device 208 has a direct visual line of sight to the pavement surface. In other cases, the edge device 208a can also be mounted to various other locations outside or within the vehicle body (e.g., front or rear vehicle bumpers, etc.). Alternatively, or additionally, the edge device can be mounted to an external object being moved by the vehicle, such as a car trailer. In still other cases, edge devices can also be mounted to air-borne objects (e.g., edge device 208b on drone 207), or on stationary objects in proximity to the pavement (e.g., edge device 208c on light pole 209). In some cases, edge devices can be mounted, for example, directly onto an individual's clothing (e.g., an inspector's jacket), or otherwise, any other object being moved by an individual along the pavement (e.g., a dolly, etc.).

As provided herein, each edge devices 208 is configured to monitor pavement condition by collecting pavement monitoring data. The pavement monitoring data can include, for example, one or more of visual data (e.g., images or image sequences, videos etc. of the pavement surface), as well as other sensor data (e.g., motion or orientation data).

In particular, in some example embodiments, the edge devices can include cameras for capturing images of the pavement surface 204. For example, as the vehicle 202 is traversing the road network, the cameras in the edge device 208a can capture one or more images of the pavement surface within an imaging area 210. In some other embodiments, the edge devices can include a video recording functionality and can record videos of the pavement surface within an imaging area 210. In at least some embodiments, the edge device can be configured to redact certain confidential information and generate redacted images/videos, and pavement distress analysis takes place on the redacted images/videos.

The edge devices 208 can also collect sensor data, such as motion or orientation data. For example, vertical acceleration data 211 (i.e., z-axis acceleration) can be monitored to record vibrations that result from the vehicle 202 driving over pavement distresses. This may provide the advantage of accurately measuring the severity of certain ride-based defects (e.g., bumps, corrugations, railroad crossing, shoving and swells).

Similarly, orientation data (e.g., gyroscope data) may also be collected by the edge device. For instance, the edge device can monitor the change in pitch 213 of a vehicle 202, which can indicate the presence of certain pavement distresses on the road (e.g., swells).

It is appreciated that not all edge devices may be configured to capture sensor data (e.g., motion and/or orientation measurements). For example, where the edge devices are mounted to stationary or air-borne objects (e.g., edge devices 208b, 208c), the edge devices may primarily rely on collecting only visual data. In other cases, some edge devices may collect sensor data, but not visual data.

The edge devices 208 can also include location sensors (e.g., GPS modules). In particular, the location sensors can generate location data for tracking the location of the edge device. In various cases, as provided herein, this can assist in localizing pavement distresses identified by the visual, motion or orientation data.

In embodiments provided herein, pavement monitoring data, collected by the edge devices 208, may be analyzed to identify the presence and/or location of pavement distresses on a pavement surface, as well as determining one or more attributes associated with the identified pavement distresses (e.g., the type of pavement distress, the severity of the pavement distress, as well as the size of the pavement distress).

In particular, in at least some embodiments, one or more trained machine learning models may be used to analyze the pavement monitoring data to determine the presence and location of pavement distresses, as well as associated pavement distress attributes (collectively referred to herein as pavement distress data). In various cases, separate trained machine learning algorithms may be provided to analyze captured image data, and motion and/or orientation sensor data, and generate corresponding pavement distress data.

In various cases, pavement distress data can be further processed to generate one or more pavement condition outputs. Pavement condition outputs indicate the condition of a pavement segment. For example, pavement condition outputs can include PCI or IRI values determined for a pavement segment.

Referring still to FIG. 2A, as shown, each of the edge devices 208 may communicate with a remote server 214 (e.g., a cloud server). In various cases, this allows the edge devices 208 to act as part of the Internet of Things (“IoT”) (i.e., IoT edge devices). Communication between the edge devices 208 and remote server 214 can occur, for example, through a wireless cellular network 212.

In at least one embodiment, the server 214 may receive raw or pre-processed pavement monitoring data from each edge device 208 (e.g., in real-time or near real-time). Server 214 may then process the pavement monitoring data to determine pavement distress data associated with one or more identified pavement distresses. For example, the server 214 may host the one or more trained machine learning algorithms which can analyze pavement monitoring data to determine the corresponding pavement distress data.

In other cases, the processing of pavement monitoring data may be distributed between the edge devices 208 and the server 214. For example, the edge devices 214 may partially process the collected pavement monitoring data, and may transmit the partially processed data to the server 214 for further processing.

Server 214 can also transmit data to the edge devices 208. For example, server 214 may transmit update configuration data to the edge devices. The update configuration data can include, for example, instructions for adjusting one or more settings (e.g., configurations) of the edge devices (e.g., the type of data transmitted by the edge devices, the frequency of data transmission, the sensitivity of various cameras and sensors (e.g., camera zoom, etc.)).

As provided in further detail herein, the automated monitoring of pavement condition by the edge devices 208 and/or the server 214 may occur in real-time or near real-time. In particular, the server 214 may receive real-time or near real-time pavement monitoring data from one or more edge devices 208. The data can then be processed to provide pavement condition outputs (e.g., real-time or near real-time) in respect of the surveyed road networks. To this end, it will be appreciated that any number of edge devices may be operating at any given time, and/or may be collecting data of the same or different road networks at the same or different times. In these cases, the server 214 may collect data from multiple edge devices and provide real-time or near real-time updates in respect of pavement condition.

In some cases, the edge device 208 may also perform an image redaction functionality. For example, the edge device can include a redaction module, which allows redaction of specific features in a captured image. In turn, only redacted media elements can exit the edge device 208 through the network 205. For example, this can include redacting privacy-sensitive material (e.g., people's faces, license plates, etc.) which is captured by the edge device camera as it images the road pavement. These redacted images can then be transmitted and analyzed, i.e., by server 214, to determine pavement distress data on the road pavement. Accordingly, the image redaction can prevent redacted, and privacy sensitive media elements from being transmitted over the network 205 to other computing devices 214 and/or server 216, which can risk the redacted media being intercepted during transmission by, for example, unscrupulous third parties. This is contrasted to many prior systems where the redaction occurs on a remote third party computing device (e.g., a server), and therefore presents the risk that the unredacted media elements can be intercepted, and the privacy sensitive information contained therein being possibly used for nefarious purposes.

It will now be appreciated, in view of FIGS. 2A and 2B, that an edge device refers to a device at the “edge” of the network 205 and which is deployed in the environment 200a to collect sensor data comprising, for example, pavement monitoring data (e.g., visual data, as well as motion or orientation data). A feature of each edge device 208 is then to transmit the collected pavement monitoring data, in either raw, partially processed (or fully processed) form, to one or more central server 214. Central server 214 may then aggregate the received data to determine, for example, pavement condition outputs.

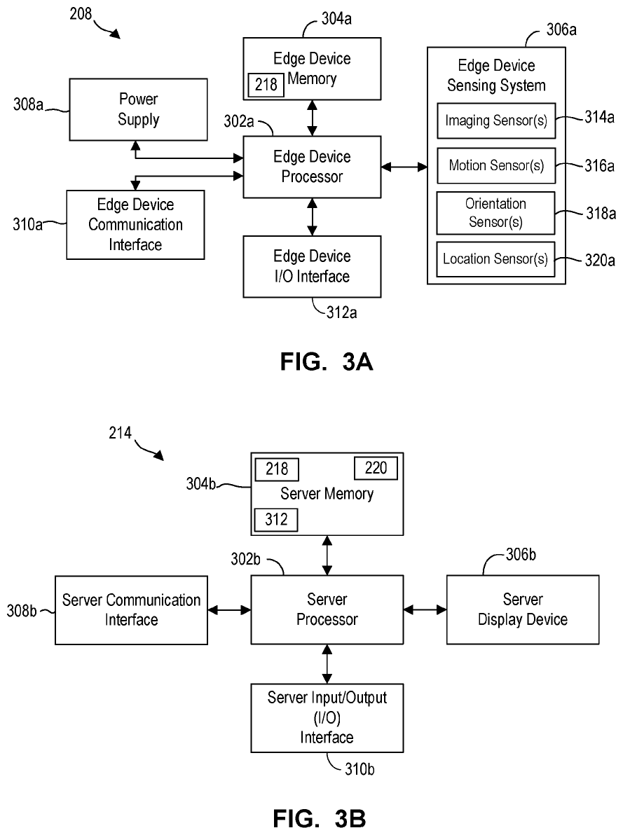

In at least some cases, the edge device 208 may not only be defined by its function in the overall system (e.g., data collection in the environment and transmitting data to server), but also by its processing capabilities. That is, edge devices 208 may be generally characterized by low processing and computational power (i.e., see edge device processor 302a in FIG. 3A). That is, the edge device 208 may not have as high computational power as server 214 (see e.g., server processor 302b in FIG. 3B). In this manner, the edge device 208 may be capable of providing limited computational abilities as compared to the server 214. For example, whereas the server 214 may be capable of processing aggregated pavement monitoring data, received from multiple edge devices 208, using high computational algorithms (e.g., machine learning models), the edge device 208 may not be similarly enabled. For this reason, the edge device 208 may only be capable of acquiring pavement monitoring data and performing simplified processing (e.g., pre-processing) before the data is transmitted to server 214 to determine pavement condition outputs. Environment 200a may also include one or more user devices 216a, 216b. User devices 216 may be any suitable computing devices, and may include mobile devices such as smartphones, tablets or laptops. In various cases, users may access raw and processed data generated by the edge devices 208 and/or server 214 directly though their user devices 216. In at least some embodiments, the user devices 216 (e.g., phones, laptops, tablets, etc.) may also act as edge devices.

In view the foregoing, the provided system and method may overcome a number of the previously-stated deficiencies associated with conventional methods for monitoring pavement condition. For example, the disclosed systems and methods do not rely on visual inspection of pavement by human inspectors, but rather, rely on processing— e.g., using one or more trained machine learning models—pavement monitoring data collected by one or more edge devices (e.g., in real-time or near real-time). Accordingly, the provided systems and methods may be able to provide more accurate indications of pavement condition (e.g., PCI, IRI, etc.) that are not otherwise subject to the same human errors or subjective assessments evident in human-based visual inspections. Moreover, by automatically monitoring pavement condition using data collected from edge devices, pavement condition is more easily monitored without users requiring specialist knowledge in respect of road infrastructure.

The systems and methods provided herein also enable monitoring of pavement condition data using lower cost, smaller form factor, plug-and-play IoT devices 208 which rely on simpler sensing systems (e.g., cameras, gyroscopes, accelerometers, LiDAR, etc.) that can determine pavement condition based on surface-level pavement analysis. More particularly, the disclosed edge devices may be easily mounted to existing infrastructure, as well as exiting fleets of vehicles that are already moving around road networks (e.g., garbage trucks or busses). This is contrasted to current systems that require specially adapted vehicles for carrying large and costly monitoring equipment, including ground penetrating radar (GPR), and which require collecting beneath pavement surface data to determine pavement condition. To this end, it is appreciated that the use of low cost, plug-and-play edge devices may assist in increasing the frequency of pavement surveys conducted by regional authorities as the expenses, time and man power associated with conventional human inspectors are eliminated, as well eliminating the cost of fitting and maintaining specially adapted survey vehicles.

Reference is now made to FIG. 2B, which shows a simplified block diagram for an example embodiment of a system 200b for automated monitoring of pavement condition, in accordance with some embodiments.

As shown, the system 200b generally includes one or more edge devices 208a-208n coupled, via network 205, to one or more of a server 214 and one or more user terminals 216. System 200b also includes a pavement distress analysis system 218, and in some embodiments, can also include a report generating system 220.

Network 205 may be a cellular network, but can also comprise one or more computer network technologies, such as IEEE 802.3 (Ethernet), IEEE 802.11 and similar technologies.

Pavement distress analysis system 218 is a system which is configured to receive pavement monitoring data (e.g., visual or other sensor data) from one or more of the edge devices 208a-208n, and can process the pavement monitoring data (e.g., in real-time or near real-time) to generate pavement distress data (e.g., data in respect of the presence and/or locations of pavement distresses, as well as associated attributes of the identified pavement distresses). As explained herein—in various cases—the pavement distress analysis system 218 can use one or more trained machine learning models to process the pavement monitoring data and generate pavement distress data. In at least some embodiments, based on the determined pavement distress attributes, the analysis system 218 can also generate one or more pavement condition outputs, including PCI values for different pavement segments.

Report generating system 220 is configured to receive either raw or processed data from the edge devices 208 and/or pavement distress analysis system 218 (e.g., in real-time or near real-time) and may, in turn, generate one or more reports of pavement condition.

For example, reporting generating system 220 can generate reports that indicate all pavement distress data determined in association with various pavement segments of a road network. In some cases, the reports can also include road network wide pavement condition. In other cases, the reports can detail severe distresses requiring urgent attention. In various cases, the reports can be generated at any variable frequency (e.g., hourly, daily, weekly, etc.).

In some cases, the report generating system 220 can receive location data from location sensors (e.g., GPS) on the edge devices. The location data can be used to localize the pavement distresses identified in the road network. A GIS software, as part of the reporting generating system 220, can then generate a visual map illustrating the location (e.g., in real-time or near real-time) of pavement distresses in the road network, along with associated attributes of each identified pavement distress. In other cases, the visual map can also illustrate the pavement condition (e.g., PCI values) of various pavement segments in a road network. Reports (or maps) generated by the report generating system 220 may be accessible by one or more user terminals 216.

While the pavement distress analysis system 218 and report generating system 220 have been separately illustrated in FIG. 2B for ease of exposition, it will be appreciated that these systems may be housed inside one or more of the edge devices 208, server 214 or user terminal 216. For example, the defect analysis system 218 may be wholly housed within each of the edge devices 208 or server 214. Alternatively, or in addition, the defect analysis system 218 may be partially housed between the edge devices 208 and the server 214. For example, using the pavement distress analysis system 218, the edge devices may partially process pavement monitoring data, and may transmit the partially processed data to the server 214. The server 214 may then continue processing the data also using the distress analysis system 218. Similarly, the report generating system 220 can be partially or wholly housed between the edge devices 208, server 214 and/or the user terminal 216.

Reference is now made to FIG. 3A, which shows a simplified block diagram of an example embodiment of an edge device 208, in accordance with some embodiments.

As shown, the edge device 208 can generally include an edge device processor 302a in communication with one or more of an edge device memory 304a and an edge sensing system 306a. In various cases, the edge device processor 302a may also couple to one or more of a power supply 308a, an edge device communication interface 310a and an edge device input/output (I/O) interface 312a.

Edge device processor 302a is a computer processor, such as a general purpose microprocessor. In some other cases, processor 302a may be a field programmable gate array, application specific integrated circuit, microcontroller, or other suitable computer processor.

Edge device processor 302a is coupled, via a computer data bus, to the edge device memory 302b. Memory 302b may include both volatile and non-volatile memory. Non-volatile memory stores computer programs consisting of computer-executable instructions, which may be loaded into volatile memory for execution by processor 302a as needed. It will be understood by those of skill in the art that references herein to an edge device 208 as carrying out a function or acting in a particular way imply that processor 302a is executing instructions (e.g., a software program) stored in memory 302b and possibly transmitting or receiving inputs and outputs via one or more interfaces. Memory 302b may also store data input to, or output from, processor 302a in the course of executing the computer-executable instructions. In various embodiments, the memory 302b can store all or some of the pavement distress analysis system 218. Edge device memory 302b may also store data generated by the edge device sensing system 306a.

Edge device sensing system 306a can include one or more devices and sensors for monitoring pavement condition and collecting pavement monitoring data. For example, the sensing system 306a can include one or more of an imaging sensor 314a, motion sensor 316a, orientation sensor 318a and location sensor 320a.

Imaging sensor 314a may be, for example, a camera for capturing media (e.g., image, image sequences or videos). In other cases, the imaging sensor 316a may be a LiDAR system, or any other system operable to capture any other type of visual “image” data. As explained herein, images generated by the imaging sensor 316a can be further analyzed to detect pavement distresses, as well as classify identified pavement distress type, severity and size.

Motion sensors 316a can include, for example, accelerometers. In various embodiments, vertical acceleration data (i.e., 211 in FIG. 2A) generated by the motion sensor 318a can also be used to identify certain ride-based pavement defects.

Orientation sensors 318a can include, for example, gyroscopes as well as other sensors configured to measure the angular orientation of the edge device. In various cases, the orientation sensor data, generated by the orientation sensor—such as pitch data (i.e., 213 in FIG. 2A)—is also used to monitor certain ride-based pavement distresses, such as swells in the pavement, which can cause a vehicle to angle upwardly or downwardly.

Location sensors 320a can include, for instance, global positioning systems (GPS)—or more generally, sensors that can track and determine the position of the edge device (e.g., on a road network). In embodiments provided herein, location data—e.g., generated by the location sensors—can be associated with pavement monitoring data received from one or more of the sensors 314a-318a in order to localize identified pavement distresses within a road network. In various cases, this can allow the report generating system 220 to generate visual maps showing the locations of variously identified defects, as well as the condition of different pavement segments around the road network.

In some cases, the sensors 306a may be housed in a single enclosure. In other cases, at least some of the sensors can be attached around the vehicle (e.g., external sensors). For example, some of the orientation sensors 318a may be attached to the vehicle wheels to obtain enhanced data with respect to pavement roughness, e.g., to determine an accurate roughness index. In other cases, the imaging sensors 314a may be attached to various parts of a vehicle (e.g., front, back or sides) and can, in some cases, be pointed straight down to avoid for tilted image distortion.

Power supply 308a can be any suitable source of power for the powering the various components of the edge device 208. In various cases, power supply 308a can be an onboard power source (e.g., one or more batteries). In other cases, the power supply 308a may be external to the edge device. For example, the edge device 208 may be connectable to a power source associated with a vehicle which houses the edge device. In various cases, the various components of the edge device 208 are configured to be powered by a low power supply (e.g., 5V to 12V), as may be available in vehicles, drones, etc. In particular, this avoids the requirement for a large dedicated power supplies, as may be the case for vehicles specially adapted with large GPR systems.

Edge device communication interface 308a is one or more data network interfaces for communicating over network 205, such as interfaces for communicating via cellular networks, as well as IEEE 802.3 or IEEE 802.11 interfaces. It will be understood that reference herein to an edge device 208 as transmitting or receiving data implies that the edge device processor 302a is transmitting or receiving data via the communication interface 308a.

Edge device input/output (I/O) interface 312a is any interface for receiving or displaying data. In some cases, the I/O interface 312a can include a display device (e.g., an LCD screen) for displaying a graphical user interface (GUI) to allow a user to modify specific parameters of the edge device 208. The display device may also receive inputs, as is the case for a touchscreen display (e.g., a capacitive touchscreen display). The I/O interface 312a may also include any other input interface, as for example, keyboards, etc.

In at least some embodiments, the edge device 208 may also include a redaction module (not shown). The redaction module can be configured to perform redaction on images and/or videos generated by the imaging sensors such that analysis of pavement distress may occur on the redacted images and/or videos.

Reference is now made to FIG. 3B, which shows a simplified block diagram of an example embodiment of a server 214, in accordance with some embodiments.

As shown, the server 214 also includes a server processor 302b coupled to a server memory 304b. In some cases, the server processor 302b is also coupled to one or more of a server display device 306b, a server communication interface 308b and a server I/O interface 310b.

Similar to the edge device processor 302a, the server processor 302b may also be a computer processor, such as a general purpose microprocessor. In some other cases, processor 302b may be a field programmable gate array, application specific integrated circuit, microcontroller, or other suitable computer processor. Further, similar to the edge device memory 304a, the server memory 304b may include both volatile and non-volatile memory. Also, it will be understood by those of skill in the art that references herein to an server 214 as carrying out a function or acting in a particular way imply that server processor 302b is executing instructions (e.g., a software program) stored in the server memory 304b and possibly transmitting or receiving inputs and outputs via one or more interface.

In various embodiments, the server memory 304b may store, wholly or partially, one or more of the pavement distress analysis system 218, as well as the reporting generating system 220. In various cases, server memory 304b can also store archived data 312. The archived data can include, for example, raw pavement monitoring data (e.g., captured images, motion and orientation sensor data, associated location data), as well as other processed data (e.g., data processed by the pavement distress analysis system 218). The archived data 312 can be stored for subsequent processing, or retrieval by the report generating system 220.

It will be understood that server 214 need not be a dedicated physical computer. For example, in various embodiments, the various logical components that are shown as being provided on server 214 may be hosted by a third party “cloud” hosting service such as Amazon™ Web Services™ Elastic Compute Cloud (Amazon EC2). As with all devices shown in the system 200a, there may be multiple servers 212, although not all are shown.

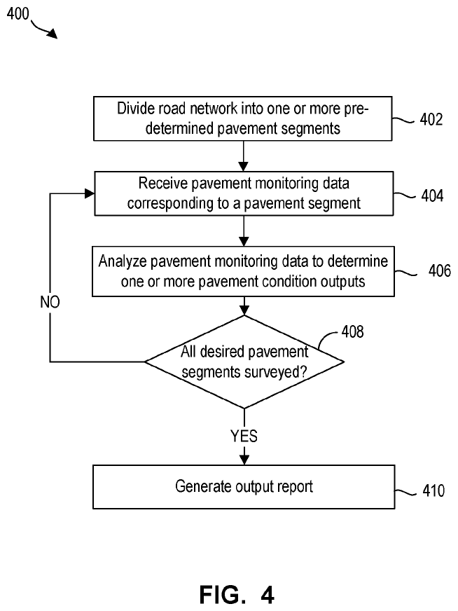

Reference is now made to FIG. 4, which shows an example embodiment of a method 400 for automatic monitoring of pavement condition, in accordance with some embodiments. Method 400a can be performed by the pavement distress analysis system 218 being executed by one or more of the edge device processor 302a and/or the server processor 302b.

At 402, the road network being surveyed is divided (i.e., segmented) into one or more pavement segments. In particular, each pavement segment may have a pre-defined length. For example, in some embodiments, each pavement segment may have a length of 20 meters. In other cases, each pavement segment may have a length of 100 meters. In still other cases, different pavement segments around a road network can have variable pre-defined lengths. In various cases, the length of the pavement segments may be determined and varied by an operator of a user terminal 218, and transmitted to the server 214. For example, the system may have knowledge of existing road network maps of a geographic location (e.g., a city), and may further receive a desired length dimension for pavement segments (e.g., from a human operator). In turn, the system may automatically generate a plurality of pavement segments with reference to the existing road network map by dividing road network map into various pavement segments based on the desired pavement segment length dimension.

At 404, in respect of any given pavement segment, pavement monitoring data is collected by one or more edge devices (e.g., in real-time or near real-time). The pavement monitoring data can include, for example, image, motion and/or orientation data collected from one or more sensors of the edge device's sensing system 306a. The pavement monitoring data may be collected by the sensing system 306a, for example, while a vehicle, carrying the edge device 208, is traversing over the given pavement segment. In some embodiments, location data from location sensor 320a is used to determine which pavement segment is being monitored by the edge device at any given time, and accordingly, which pavement segment should be associated with the received pavement monitoring data.

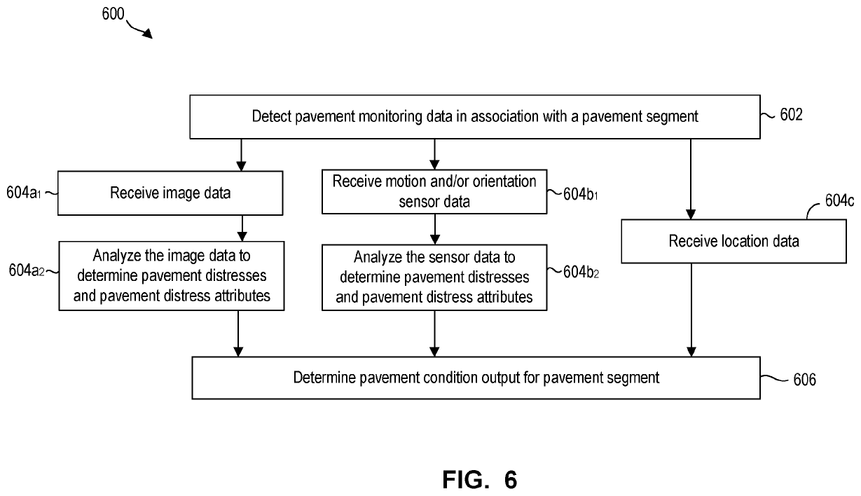

At 406, the pavement monitoring data is analyzed to determine one or more pavement condition outputs in association with a pavement segment. For example, the pavement condition outputs can comprises PCI values, or otherwise, any other measurement of road condition (e.g., an IRI value, or a Pavement Surface Condition (PSC) rating) in association with a pavement segment. The method of analyzing pavement monitoring data to determined pavement condition outputs is explained in greater detail herein with reference to FIGS. 6-11.

At 408, it is determined whether all pavement segments desired to be surveyed in the road network have been surveyed. For example, this is determined by analyzing whether pavement monitoring data is received in association with each pavement segment. If this is not the case, the method 400 may return to act 404 to receive further pavement monitoring data (e.g., in real-time or near real-time) in respect of other pavement segments in the road network. For example, this can correspond to the next pavement segment on the vehicle's driving path, as the vehicle travels through the road network.

Otherwise, if all pavement segments have been surveyed, then in some embodiments, at 410, an output report may be generated. For example, the output report may be generated by the report generating system 220 as explained previously.

For example, as previously stated, the output report may be, for instance, a report detailing road network wide pavement condition values (e.g., PCI, IRI, PSC, etc.) for each pavement segment in the road network. In other cases, the report may be a report that details locations of various pavement distresses that require urgent attention. For example, if the PCI value of a pavement segment is above a pre-determined threshold—or a pavement distress is determined to have a high severity—it may be included in the urgent attention report.

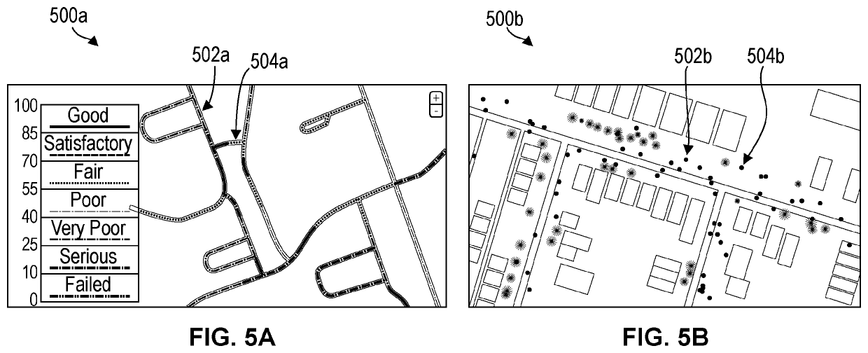

In other cases, the report generated at act 410 may be a visual report. FIG. 5A illustrates an example road network map that includes PCI values determined for each pavement segment (i.e., 502a, 504a) in the road network. FIG. 5B illustrates another example road map 500b comprising the locations of various pavement distresses (i.e., 502b, 504b) within the road network. The road network maps 500a, 500b may be generated using, for example, a GIS software operating on the server 214. In particular, the GIS software can generate the maps by combining location data from the location sensor 320a, with determined pavement condition outputs.

Although the method 400 illustrates the output report as being generated once all pavement segments are surveyed, it will be appreciated that, in other embodiments, the output report can be generated—as well as updated—on a rolling basis as new pavement condition outputs are being determined (e.g., in real-time or near real-time). That is, it is not necessary to wait until all pavement segments are surveyed before populating, generating or updating the report.

In at least some embodiments, the reports may be generated and updated in based on pavement monitoring data received from the multiple edge devices 208 deployed on the road network. For example, if more than on edge device is deployed on the road network, the report can be updated based on most recent pavement monitoring data received from an edge device. In other cases, pavement condition outputs, determined from data from different edge devices, can be aggregated into a single output as, for example, by averaging the different outputs.

Referring now to FIG. 6, there is shown an example embodiment of a method 600 for automatic monitoring of pavement condition for a pavement segment, in accordance with some embodiments. More particularly, method 600 discloses, in further detail, the process of analyzing pavement monitoring data at act 406 in method 400 in order to determine one or more pavement condition outputs. Method 600 may be performed, for example, by the pavement distress analysis system 218 being executed by one or more of the edge device processor 302a and the server processor 302b.

As shown, at 602, the edge device processor 302a may monitor for any pavement monitoring data that may be received in association with a pavement segment. The pavement monitoring data can include, for example, data generated by one or more sensors of the edge device's sensing system 306a. The pavement monitoring data may be generated as a vehicle, carrying the edge device 208, is traversing over the pavement segment.

At 604, the edge device processor 302a can receive the pavement monitoring data in association with the pavement segment. The received pavement monitoring data can include, for example, one or more of image data generated by the imaging sensor 314a (604a₁), motion and orientation data generated by the motion and orientation sensors (604b₁) and/or location data generated by the location sensor 320a (604c). The image data, motion and orientation data as well as the location data may be received concurrently, or partially concurrently, by the edge device processor 302a. For example, as a vehicle is travelling over a pavement segment, various image, sensor and location data may be generated by the sensing system 306a.

As shown, method 600 can accordingly branch into one or more parallel branches that occur concurrently, or partially concurrently, and correspond to receiving and analyzing image data (604a), receiving and analyzing motion and orientation data (604b), and receiving location data (604c).

In respect of the first branch 404a—at 404a₁, the edge device processor 302a may receive image data, associated with a pavement segment, from the image sensor 314a. For example, the image data may comprise a single image frame, or a plurality of image frames captured by the image sensor 314a at pre-defined time or frequency intervals. The image data can comprise, for instance, one or more of photographs captured by a camera, digital video or other image-type data (e.g., LiDAR data).

At 604a₂, the image data is analyzed to identify the location of any pavement distresses in the captured images, as well as determining one or more attributes of the identified pavement distresses (e.g., type of distress, severity, size). The analysis of the image data may occur either on the edge device processor 302a and/or the server processor 302b. In at least one embodiment provided herein, the image data may be analyzed using one or more trained machine learning models configured to analyze the image data to identify imaged pavement distresses and determine the one or more associated pavement distress attributes.

Similarly, with respect to the second branch 604b—at 604b₁, the edge device processor 302a can also receive motion and orientation data, associated with the same pavement segment. The motion and orientation data can be received from the motion sensor 316a and/or orientation sensor 318a. At 604b₂, the motion and/or orientation sensor data is also analyzed to also identify one or more pavement distresses in the collected data, as well as determining attributes associated with the identified pavement distresses (e.g., type of distress, severity and size). In particular, as previously explained, the motion and orientation data can be used to identify and classify specific ride-based distresses in accordance with ASTM 6433 standards (e.g., bumps, corrugations, railroad crossing, shoving and swells), which are classified based on acceleration and rotation data.

In at least some embodiments, the motion and/or orientation data may be also analyzed using one or more trained machine learning models configured to analyze the sensor data to locate pavement distresses and identify one or more associated pavement distress attributes. Analysis of the motion and/or orientation data may occur either on the edge device processor 302a and/or the server processor 302b.

Edge device processor 302a can also receive location data from the location sensor 320a, at 604c. In at least some cases, the location data may be associated with each of the image, motion and orientation data to assist in localizing the exact (or relative) position of identified pavement distresses within a road network. As previously explained, this can be used by the report generating system 220 to generate one or more visual maps of the of the pavement distresses.

At 606, the output of each of the analysis blocks 604a2, 604b₂—along with raw pavement monitoring data 604a₁, 604b₁ and 604c—may be further processed to determine one or more pavement condition outputs associated with the pavement segment. For example, this can include determining various pavement condition values, including PCI, IRI, etc.

In some cases, it may be possible to combine pavement monitoring data, associated with same pavement segment—but which are collected at different times or by different edge devices.

For example, in some cases, “geofences” can be defined on the road network map. A geofence can represent a geographic area on the road map (e.g., a square). The size of the geofence can be based on the accuracy of the GPS data being collected. For example, the lower the accuracy of the data, the larger the size of each geofence area. In some cases, the geofence area may be approximately 10×10 meters.

Each previously detected pavement defects may be associated with a corresponding geofence, based on the location of the detected defect. Accordingly, when the same pavement segment is re-monitored (e.g., due to multiple drive-bys)—the system may: (i) initially, determine the geofence areas associated with that pavement segment; (ii) detect a pavement defect in at least one geofence area; (iii) determine if a pavement defect was previously detected in the same geofence area; (iv) if so, then the data in respect of the new detected pavement defect can either be discarded, used to update the previously located pavement defect, or averaged with the previously acquired pavement monitoring data; and (v) otherwise, if no pavement defect was previously detected in the same geofence area, then the new data—in respect of the newly detected pavement defect—can be used to update the system to identify a new defect in the pavement. In turn, this allows catching defects that were missed on a first pass, without duplicating defects that are noticed on multiple passes.

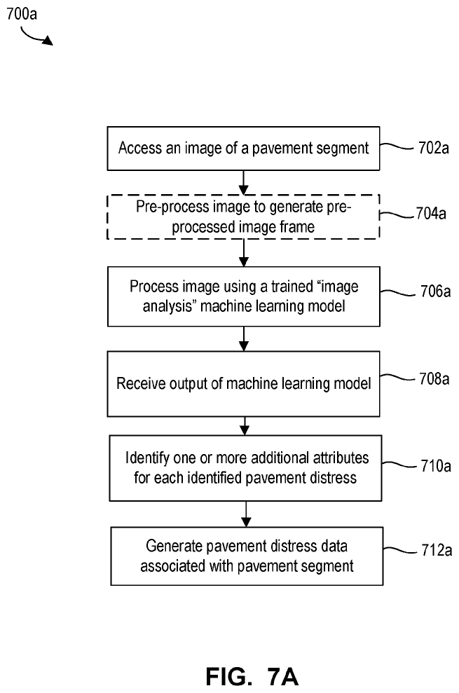

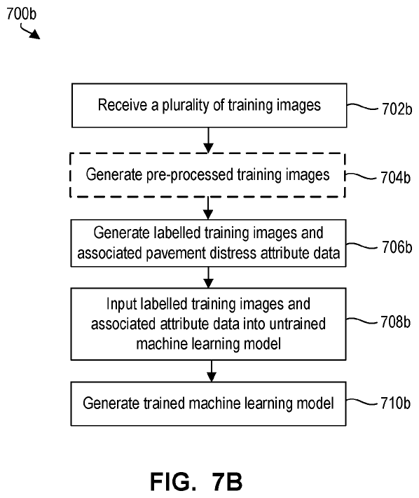

Reference is now made to FIGS. 7A and 7B, which show example embodiments of methods for analyzing image data to monitor pavement condition of a pavement segment.

In particular, method 700a of FIG. 7A shows an example method for using trained machine learning models to analyze image data of pavement segments to identify the presence of pavement distresses, as well as to identify one or more attributes of these pavement distresses. Method 700b of FIG. 7B shows an example method for training the machine learning model used in method 700a to analyzes input image data. Methods 700a and 700b can be executed, for example, by one or more of the edge device processor 302a and server processor 302b.

Referring first to FIG. 7A, which shows an example embodiment of a method 700a for using a trained machine learning model to process image data to determine the pavement condition of a pavement segment. Methods 700a can correspond to the analysis performed at act 604a2 in FIG. 6.

As shown, at 702a, an image captured of a pavement segment is accessed. For example, where the method 700a is executed on the edge device processor 302a, act 702a can involve accessing an image received in real-time or near real-time from the image sensor 314a. In other cases, a previously captured image may be stored in the edge device memory 304a and accessed from the edge device memory 304a.

In other cases, act 702a may be performed by the server processor 302b. For example, the server processor 302b may receive, via network 205, images from an edge device 208 (e.g., in real-time or near real-time). The images may be accessed immediately, or otherwise stored on the server memory 304b and subsequently accessed.

At 704a, the image frame may undergo one or more pre-processing operations to generate a pre-processed image frame. For example, this can include converting the image to grayscale and/or down sampling the image. In some cases, the images can be downscaled to a 640×480 pixel image. In various cases, the pre-processing operations can allow the trained machine learning model to more efficiently analyze the image.

In at least some embodiments, the pre-processing at 704a is performed by the edge device processor 302a before transmitting the image to the server 214 for further analysis.

At 706a, the image, or pre-processed image, is fed into a trained image analysis machine learning model. The trained machine learning model is configured to analyze the image to locate pavement distresses, as well as identify one or more attributes of the identified pavement distresses (e.g., classifying the type and severity of each identified pavement distress).

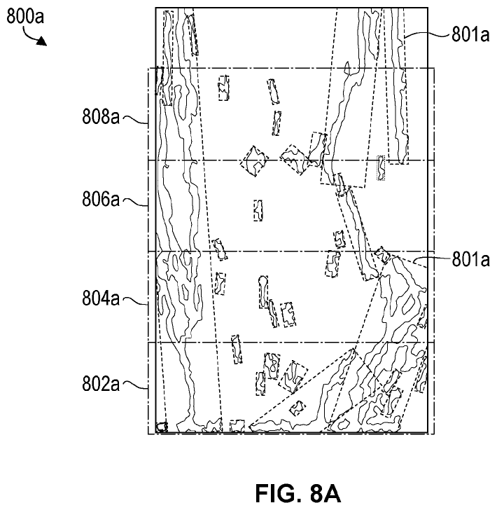

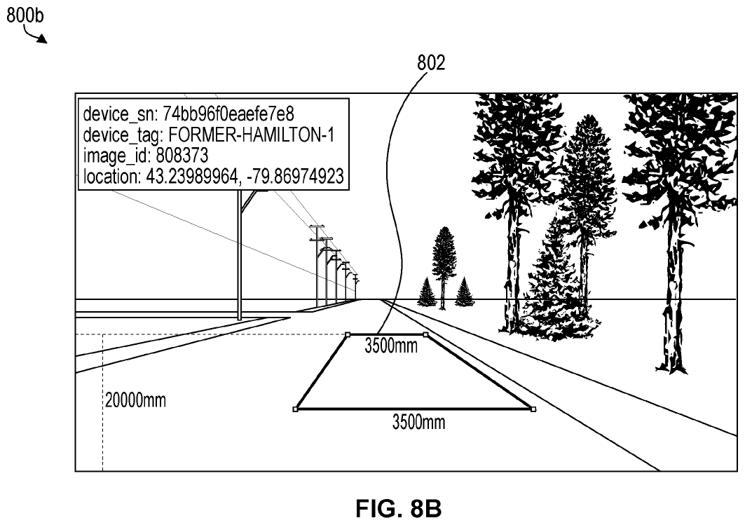

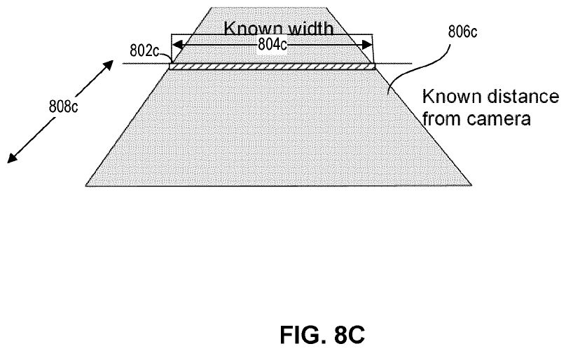

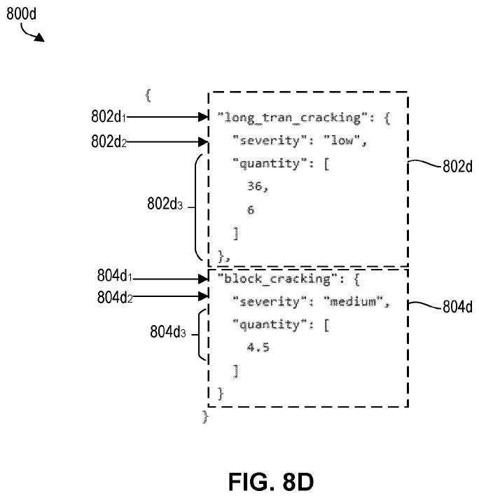

In some embodiments, the output of the trained machine learning model is an image comprising one or more visual indications of the location of the pavement distress in the image. For example, the trained machine learning model can output a labeled image having a visual bounding box delineating the area which includes the pavement distress (e.g., bounding boxes 801a in FIG. 8A, or 802b in image 800b of FIG. 8B). In other cases, the location of the pavement distress can be identified in any other manner. For example, the trained model can generate image pixel coordinates where the pavement distress is located in the image. The output of the learned model can also include data (e.g., metadata) classifying the type and severity of one or more identified pavement distresses.

In various embodiments provided herein, the trained machine learning model is a trained convolution neural network (CNN) configured to perform feature extraction and classification on input images of pavement segments.

At 708a, the outputs from the trained machine learning model are received.

At 710a, the image may be further processed to determine one or more further pavement distress attributes for pavement distresses identified by the machine learning model.

For example, this can include determining the size or length of the pavement distress located within the bounding box in the labelled image. For instance, in FIG. 8B, the size or area of the pavement distress within the bounding box 802 can be determined. As explained previously, the size of the pavement distress can be used to determine the PCI of a pavement segment. In various cases, the size or area of a pavement distress is determined at 710a using various image processing techniques, including using various edge detection algorithms as are known in the art (e.g., Sobel, Canny, Prewitt, Roberts and fuzzy logic methods).

In at least some embodiments, prior to determining the further attributes of the pavement distresses at 710a (e.g., pavement distress size), the image may undergo one or more image adjustment operations.

For example, as shown in FIG. 8B, the image of the pavement segment may be slightly distorted owing to the placement of the edge device (e.g., on a windshield). More particularly, owing to the elevated and/or titled placement of the edge device, the edge device may not capture a top-down image of the pavement segment (e.g., FIG. 8A), but rather may capture photographs of the pavement segment at a slight tilted elevation angle (e.g., FIG. 8B). In this distorted image, distances farther away from the camera are occupied by fewer pixels for the same width and depth of the road. This, in turn, may prevent accurate estimation of the pavement distress length or size, as the pavement distress may appear more elongated in an angled image.

Accordingly, to ameliorate this concern, at act 710a, the image may be adjusted to compensate for any distortions. For example, image processing software (e.g., stored on the edge device memory 304b or server memory 304b), may apply one or more image adjustments to compensate for angled and/or titled image views. More specifically, the image processing software may adjust the image based on the known angular and elevation placement of the edge device 208 (e.g., on a vehicle's windshield).

In some cases, the angular and elevation placement of the edge device may be provided beforehand by a user, either through a GUI on the edge device's display screen (e.g., I/O interface 312a), or on a GUI on a user terminal 218 in communication with server 214. In other cases, at least the angular position of the edge device may be determined from orientation data generated by the orientation sensors 318a.

In some cases, the tilt in the image can be compensated by applying an initial calibration calculation. For example, as shown in FIG. 8C, prior to performing method 700b— an object 802c, with a known width 806c, may be placed on a pavement section 804c, at a known distance 808c from the edge device 208 (e.g., the edge device's camera). For example, the object 802c may be a ruler or a calibration mat. In the case of a calibration mat, the mat may itself extend all or part of the distance 808c. The edge device camera is then operated to capture an image of the pavement segment 806c. The captured image is then used to calculate the distortion in the image (e.g., tilt and elevation) based on the calibration points generated using the known width and distance placement of object 802c. For example, this can involve calculating a transformation matrix that transforms the distorted image into a top-down undistorted view of the pavement segment (FIG. 8A). This process functions as an acceptable way of estimating the distortion because the road surface (where all defects are found) can be assumed to be level. In particular, even when the road is not perfectly even or level, this estimation is more than adequate to allow us calculation of the surface area or length of features for the purpose of index calculations.

In some embodiments—rather than performing act 710a separately from act 708a—the trained learning model itself may be configured (i.e., trained) to also analyze images to automatically determine pavement distress quantities and sizes.

At 712a—pavement distress data associated with the pavement segment—is generated. In particular, the pavement distress data can comprise a summary of each located pavement distress in the image, as well as all associated attributes of that pavement distress (e.g., type, severity, size, etc.).

For example, FIG. 8D shows an example output file 800d that can include pavement distress data generated at 712a. In this example, the output is a PCI JSON file that includes one or more data elements, corresponding to different types of identified pavement distresses in the image, and associated attributes. In the illustrated example case, the JSON file includes a first data element 602d corresponding to a first identified longitudinal traversal crack, and a second data element 604d corresponding to a second identified block crack. Each of the first and second data elements 602d, 604d includes a respective field 602di, 604di identifying the defect type, a field 602d2, 604d2 identifying the severity of that defect (e.g., high, low, medium), and a field 602d3, 604d3 identifying the size of that defect. As explained in further detail herein, the JSON file can be used to determine one or more pavement condition outputs (e.g., PCI for the pavement segment, etc.).

Referring now to FIG. 7B, there is shown an example embodiment of a method 700b for training an image analysis machine learning model to analyze images of pavement segments, in accordance with some embodiments.

At 702b, a plurality of training images are received which include images of various pavement distresses on pavement segments. The plurality of training images can include, for example, images of different types of pavement distresses (e.g., in accordance with ASTM 6433 standard). Additionally, in various cases—for each type of pavement distress—the training images can also include images of the pavement distress at different levels of severity (e.g., low, medium high), different sizes of the pavement distress, images of the pavement distress captured under different lighting or climate conditions (e.g., sunny, cloudy, rainy), at different locations along a pavement segment (e.g., edge, wheel line, center, etc.), and for different types of pavements (e.g., parking lots, highways, city streets, etc.). Still further, for each type of pavement distress, the training image set can include images captured at different zoom levels (e.g., 0.5× to 2×), and at different height elevations and/or titled angles. In this manner, the training set may include a broad range of training images to generate a more highly trained machine learning model capable of locating and classifying different pavement distresses in images under a wide range of conditions.

At 704b, in some embodiments, each of the training images can be pre-processed. For example, this can include converting the training images into greyscale and/or downscaling the images (e.g., downscaling to 640×480 pixels).

At 706b, the training images may be further processed to generate a plurality of labelled training images. For example, each training image can be annotated (e.g., labelled) with the location of the pavement distress in the image. For instance, this can include drawing a visual indicator around the pavement distress in the image (e.g., drawing a bounding box), or otherwise, specifying the image coordinates where the pavement distress is located in the image. As well, for each pavement distress in each training image, one or more associated attributes can be identified, including classifying the type of image pavement distress and the severity of that pavement distress.

In various cases, act 706b may be performed by a user, such as an operator of the user terminal 216. For example, a user interacting with the user terminal 216 may be presented with various training images on a display of that user terminal, and can use an input interface (e.g., display touchscreen, keyboard and mouse, etc.), to appropriately label each training image with the location of the pavement distress. The user can also interact with the user terminal 216 to provide associated attribute data for each identified pavement distress. In various cases, the user who is processing the training images may be a skilled technician who is familiar with the various classification standards (e.g., the ATSM standard), and in particular, the various criteria for classifying the type and severity of each pavement distress. In this manner, the training images may be appropriately labelled and annotated.

At 708b, the labelled training images and associated pavement distress attribute data are fed into an untrained machine learning model, and are used to train the machine learning model. As explained in greater detail herein, the machine learning model may be a convolution neural network (CNN).

At 710b, in the machine learning model is trained to generate a trained machine learning model that is configured to analyze new input images of pavement segments to identify the location of pavement distresses in these images, as well determining associated attribute data (e.g., type and severity of distresses).

In various cases, once a trained machine learning model has been generated, the trained model may be further trained over time to enhance its predictive accuracy. For example, this can involve feeding new images into the trained model and observing the model's predicted output. If the model's output is incorrect (e.g., pavement distresses were improperly detected), the predicted outcome is identified and corrected (e.g., by a user of the user terminal 216) for the benefit of re-training the model.

In at least some embodiments, the method 700b may be performed using the server processor 302b. Once the server generates a trained machine learning model, the trained model may be “pushed out” (e.g., transmitted via network 205) to one or more edge devices 208. In this manner, the trained machine learning model may be stored on each edge device's memory 304a, therefore allowing analysis of new input images to occur directly on the edge devices. In other cases, the trained machine learning model may be simply housed on the server 214, such that all image analysis occurs on the server 214.

In view of the foregoing, methods 700a and 700b allow for automated pavement distress identification and classification in images of pavement segments using trained machine learning models. This, in turn, avoids the necessity of manual visual surveys of pavement segments by, e.g., human inspectors.

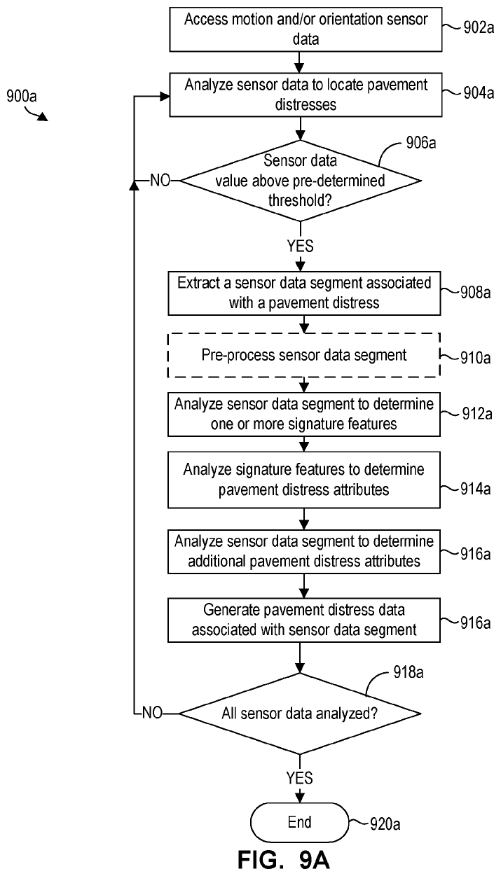

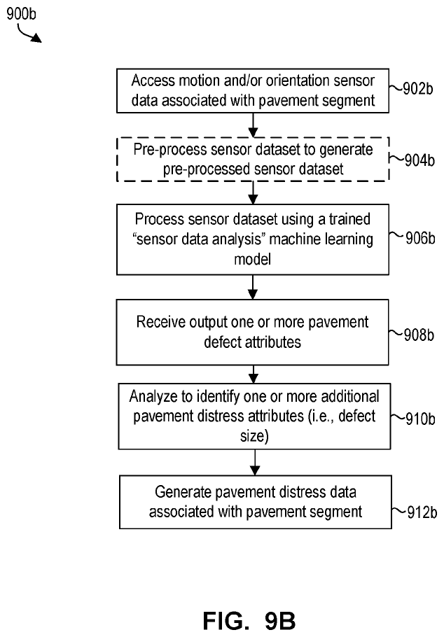

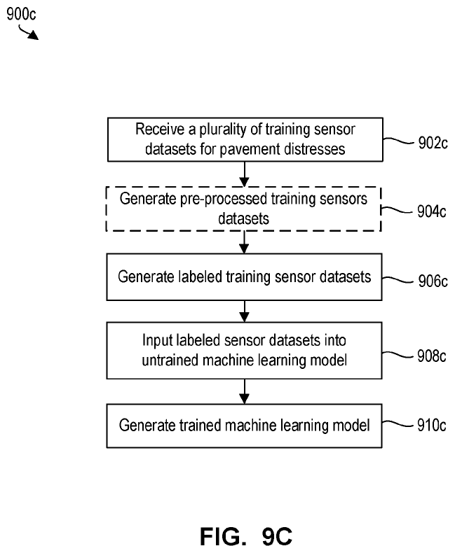

Reference is now made to FIGS. 9A to 9C, which show various example embodiments of methods 900 to 900c for automatic detection of ride-based pavement distresses using motion and orientation data. Methods 900a and 900b can correspond to the analysis performed at act 604b₂ of FIG. 6. In various cases, methods 900a to 900c can be executed, for example, by one or more of the edge device processor 302a and server processor 302b.

Referring first to FIG. 9A, which shows an example embodiment of a method 900a for processing motion and orientation data (e.g., accelerometer and gyroscope data) to identify ride-based pavement distresses in a pavement segment, according to some embodiments.

At 902a, motion and orientation sensor data associated with a pavement segment is accessed. The motion data can include, for example, accelerometer data (e.g., vertical acceleration data 211 in FIG. 2A), generated by an accelerometer 316a of the edge device 208. Orientation data can include, for example, gyroscope data (e.g., pitch data 213 in FIG. 2A), generated by a gyroscope of the edge device 208.

The sensor data can be accessed directly by an edge device processor 302a in real-time or near real-time, or may otherwise be transmitted to the server processor 302b from an edge device 208 (e.g., in real-time or near real-time). In other cases, the accessed sensor data may be archived data that was previously collected and stored on memory (e.g., edge device memory 304a, or server memory 304b).

At 904a, the sensor data is analyzed to identify the presence of pavement distresses. More particularly, it has been appreciated that for certain ride-based pavement distresses, the distresses are detectable through changes in vertical acceleration, as well as angular pitch orientation.

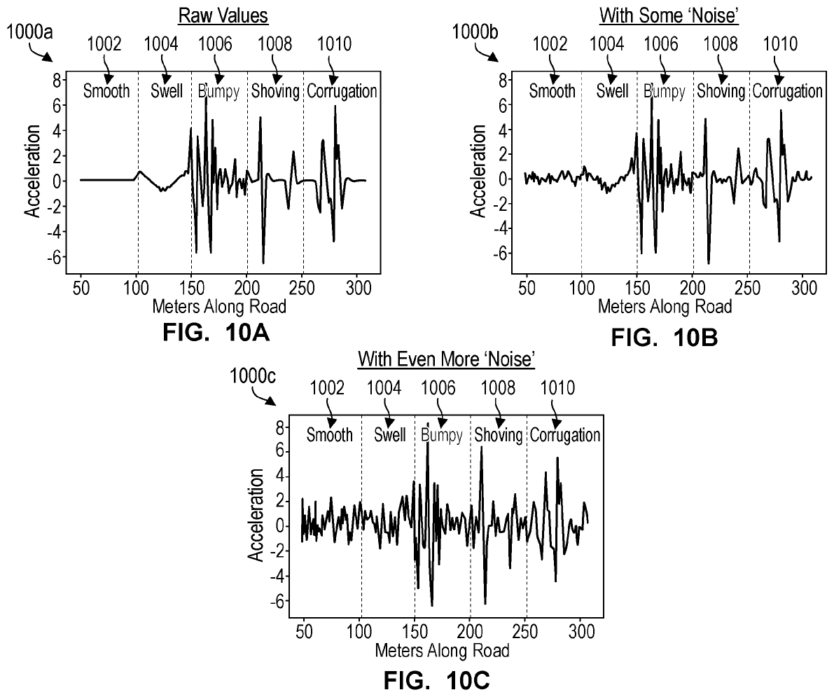

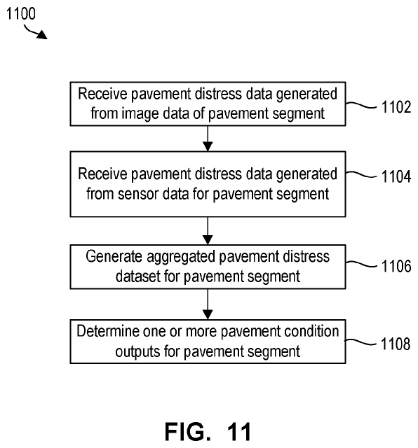

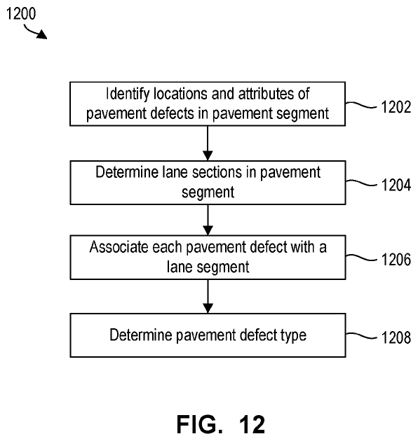

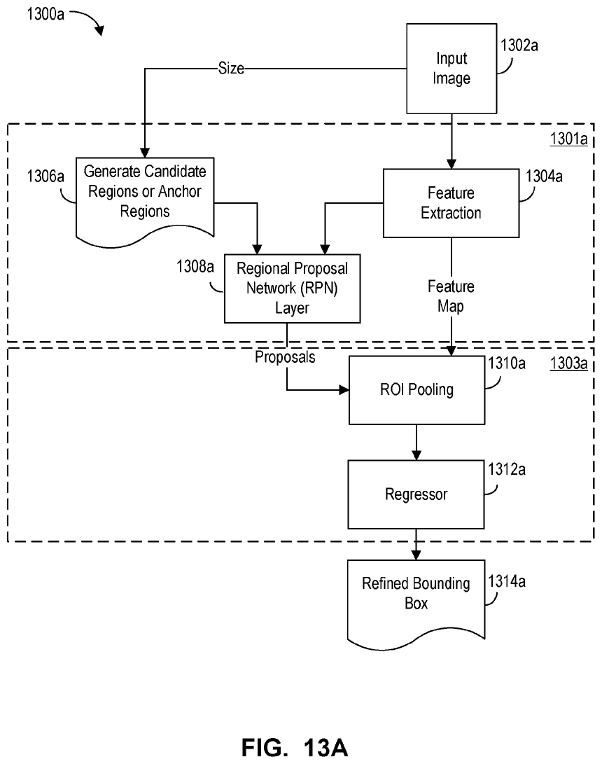

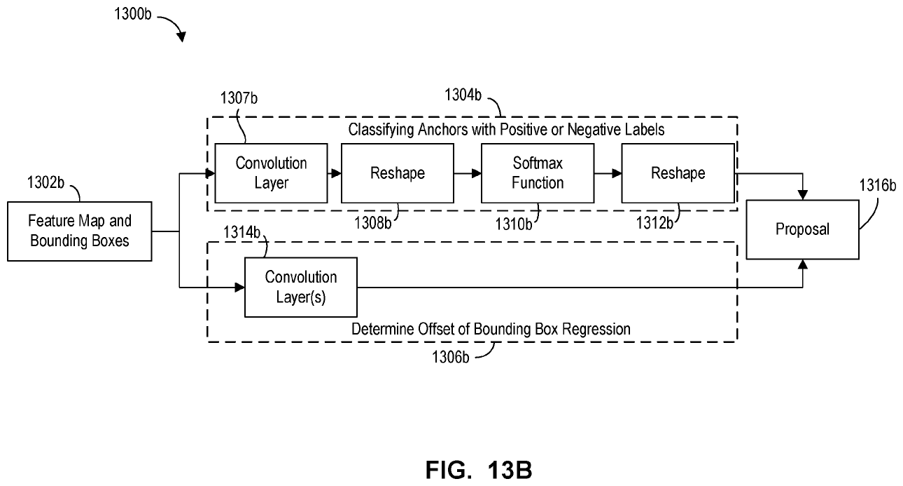

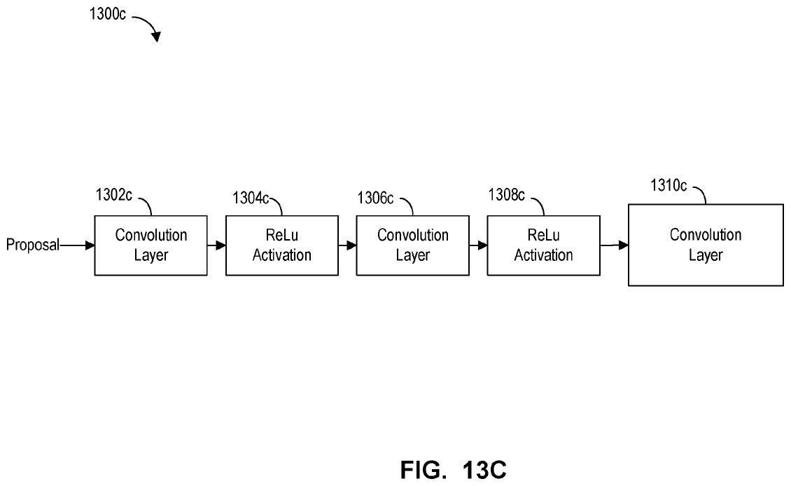

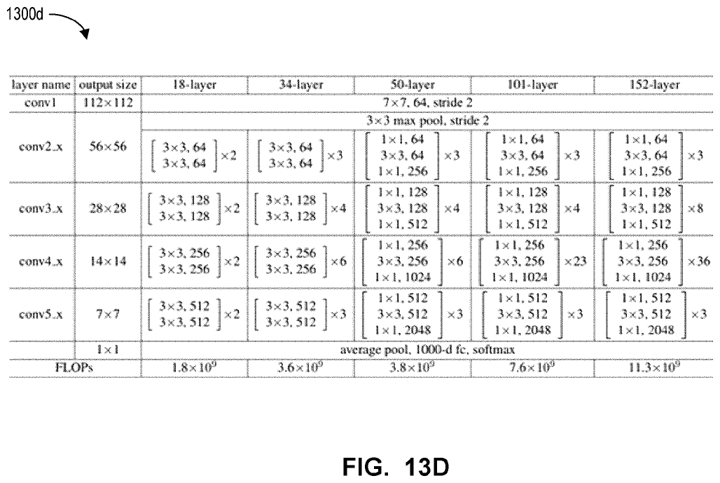

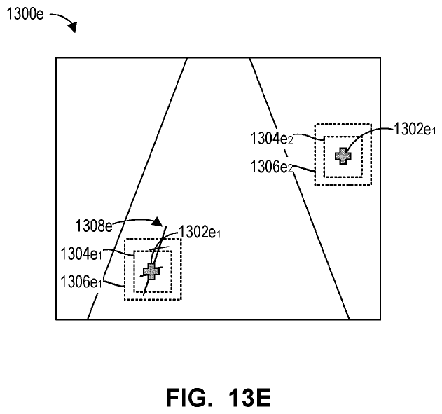

For instance, referring briefly to FIGS. 10A-10C, there is shown various example plots 1000a, 1000b and 1000c illustrating changes to vertical acceleration in response to a vehicle travelling over smooth surfaces 1002, as well as over swells 1004, bumps 1006, a shoving 1008 and corrugations 1000. More particularly, plot 1000a illustrates a vertical acceleration response having no noise, plot 100b illustrates the vertical acceleration response 900b having some noise interference, and plot 1000c illustrates the vertical acceleration response having significant noise interference. As shown, different ride-based pavement distresses demonstrate different vertical acceleration responses (e.g., varying amplitude of peaks, number of peaks, time distances between consecutive peaks, etc.).

At 906a, during the monitoring of sensor data (e.g., motion and orientation data), it is determined whether the sensor values have exceeded a pre-determined minimum threshold value. In particular, this can indicate the presence of a pavement distress in the sensor data set.

For example, motion data (e.g., vertical acceleration data) can be monitored to determine whether the motion data has exceeded a minimum pre-determined threshold. Similarly, orientation data (e.g., pitch angle data) is also separately monitored to determine whether it has exceeded a minimum pre-determined threshold.

In some cases, the pre-determined threshold can refer to an absolute data value (e.g., a pre-determined acceleration value, or otherwise, a pre-determined pitch angle value). In other cases, the pre-determined threshold may be expressed in more relative terms. For example, at 906a, it can be determined if the motion or orientation data has exceeded a minimum “standard of deviation” threshold. In particular, this can indicate a sudden change in the motion or orientation data. In some embodiments, the standard of deviation may be calculated on a rolling-basis, and the determination at 906a is satisfied if the rolling standard of deviation exceeds the minimum threshold.

In various cases, different pre-determined thresholds can be selected for different types of sensor data. For example, different minimum threshold values (e.g., standard of deviation thresholds) can be selected for acceleration data versus rotation data. In some cases, the edge device memory 304a and/or server memory 304b may store a look-up table of different thresholds for different types of sensor data. Accordingly, at 906a, an initial determination may be made as to the applicable threshold, having regard to the type of sensor data being analyzed

Additionally, minimum thresholds can also vary having regard to other factors that include, for example, the vehicle speed, vehicle weight, as well as road conditions (also referred to herein as threshold adjusting factors). For example, the vertical acceleration response may vary between heavier and lighter vehicle, as well as faster and slower vehicles. For instance, the vertical acceleration may be more dampened for heavier vehicles and/or vehicle's travelling at faster speeds. Accordingly, it may be necessary to calibrate the vertical acceleration threshold for detecting a pavement distress based on vehicle weight and/or speed. In various cases, the look-up table stored in the edge device memory 304a and/or server memory 304b can also store correlation data between different thresholds based on different threshold adjusting factors.

In various embodiments, the pre-determined threshold may be determined experimentally beforehand through experimentation, and can be stored on the edge device memory 304a and/or the server memory 304b (e.g., in a look-up table, along with the threshold adjusting factors). The pre-determined thresholds and any threshold adjusting factors can also be updated or modified, such as by a user operating a user terminal 216. For example, the threshold and/or adjusting factors can be updated on the server memory 304b, and in some cases, the updates can be pushed to different edge devices for storage on edge device memories 304a.

At 906a, if the sensor data is not above the pre-determined threshold, the method 900a can return to act 904a to continue analyzing the different sensor datasets. The method 900a may then iterate until the condition at act 906a is satisfied.

Otherwise, if the sensor data has exceeded the pre-determined threshold, at 908a, a sensor data segment, associated with the pavement distress, is identified and extracted. In other words, at 908a, the portion of data generated as a result of the pavement distress is identified (e.g., data segments 1004, 1006, 1008 and 1010 in FIGS. 10A-10C). The extracted data segment can, for example, correspond to one or more peaks in the vertical acceleration data, or one or more changes in the angular pitch data. More particularly, in various embodiments, the sensor data segment can correspond to the portion of data where the sensor values are continuously above the pre-determined threshold at 906a. For example, where the acceleration or angle data is continuously above the threshold standard of deviation. In various cases, the sensor data segment can correspond to a few milliseconds of data

At 910a, in some embodiments, the extracted sensor data segment is pre-processed. For example, this can include applying one or more noise-reduction filters to assist in better analyzing the sensor data segment.

At 912a, the sensor data segment is further analyzed to determine (e.g., extract) one or more signature features. As explained herein, the signature features can assist in classifying the type and severity of the pavement distress represented in the sensor data segment.

The types of signature features determined at 912a may vary based on the type of sensor data analyzed. For example, for vertical acceleration data, the determined acceleration signature features include one or more of the average standard of deviation of the acceleration data over the course of the data segment, the average acceleration peak height, the number of peaks, the average time distance between acceleration peaks, the time period for the acceleration data segment, the rate of increase (or decrease) of acceleration values. In some cases, one or more transformation may be applied to the sensor data to determine the signature data. For example, the acceleration data may be converted into the frequency domain (e.g., via a Fast Fourier Transform (FFT)) to determine the value of the frequency peaks.

Similarly, in respect of orientation data (e.g., pitch angle), determining angular signature features can include determining, for example, the value of the angular orientation, the rate of change of orientation, the distance between consecutive changes in angular orientation change (e.g., as the vehicle is driving up and over the pavement distress). Similarly, one or more data transforms can be applied to extract further signature features (e.g., FFTs, etc.)

At 914a, the signature features determined at 912a are analyzed to determine one or more pavement distress attributes, including classifying the type and severity of the sensed pavement distress.

In various cases, in order to determine the pavement distress attributes, the extracted signature features are analyzed in view of one or more pre-determined attribute classification rules. In particular, the attribute classification rules can stipulate requirements for correlating different signature features to different types and/or severities of pavement distresses.

For example, in respect of vertical acceleration data, an example classification rule may be that the acceleration data corresponds to a corrugation distress if the extracted signature features indicate four to five acceleration peaks detected within a 50 millisecond time period, and the standard of deviation for the data segment is above a first pre-determined threshold.

Similarly, a second classification rule may be that vertical acceleration data corresponds to a railway track if the extracted signature features indicate two distinct acceleration peak fluctuations spaced within a few milliseconds apart.

The classification rules can also be used to determine the severity of the distress. For example, if the acceleration peaks are within certain amplitude ranges—or the standard of deviation is within the certain ranges—the severity can be appropriately classified as either low, medium or high.

In some cases, the classification rules may also consider one or more external factors, such as the vehicle speed. For example, if no change in the vehicle speed was detected, this may indicate a low severity distress. Alternatively, if a notable decrease in vehicle speed was detected, this can indicate a high severity distress.

Accordingly, different classification rules can be pre-determined for different types of sensor data, and in respect of classifying the type and/or severity of the pavement distress.

At 916a, the sensor data segment can be further analyzed to determine the pavement defect size. For example, the size of the defect can be estimated by calculating the time period of the sensor data segment, multiplied by the vehicle speed. This can provide the distance travelled by the vehicle over the course of the sensor data segment, and therefore can identify the pavement defect size.

At 918a, pavement distress data, in association with the sensor data segment, is generated. For example, this maybe a JSON file as shown in FIG. 8D.

At 920a, it is determined whether all of the sensor data has been analyzed. If not, the method 900a can return to 902a and iterate until all of the sensor data has been analyzed. Otherwise, at 920a, the method 900a may end in respect of analyzing the sensor data for the specific pavement segment.

Referring now to FIG. 9B, there is shown an example embodiment of a method 900b for processing sensor data to determine pavement condition, according to some other embodiments. In contrast to the method 900a, method 900b can use a trained machine learning algorithm to analyze the sensor data.

At 902b, the motion and/or orientation sensor data is accessed. At 904b, in some cases, the sensor dataset is pre-processed, such as by applying one or more noise filters. At 906b, each sensor dataset is input into a trained machine learning model that is configured to analyze the sensor dataset to identify the presence and location of one or more pavement distresses, as well as one or more attributes of the identified pavement distresses (e.g., severity and type). In at least some embodiments, a different trained machine learning model can be used to process each type of sensor data. For example, a first trained machine learning model can be used to analyze motion data (e.g., vertical acceleration data), while a second trained machine learning model can be used to analyze rotation data (e.g., angular pitch data).

At 908b, the outputs are received from the one or more trained machine learning models. In various cases, the outputs can include indications of which time segments in the sensor datasets correspond to pavement distresses (e.g., the sensor data segments corresponding to the pavement distresses), as well as information about attributes associated with these identified pavement distresses (e.g., type and severity, etc.).

At 910b, in some cases, further attribute data can be determined for each identified pavement distress in the sensor data. For example, this can include determining the size of the pavement distress, as previously explained in relation to act 916a of method 900a.

At 912b, pavement distress data can be generated in association with analyzed pavement segment (e.g., a PCI JSON file comprising identified pavement distresses, as well as related attributes).

Referring now to FIG. 9C, which show an example embodiment of a method 900c for training a machine learning model to analyze sensor data (e.g., motion or orientation data to determine pavement condition.

At 902c, a plurality of training sensor datasets are received, wherein the sensor datasets capture sensor data responses to various types of pavement distresses (e.g., ride-based pavement distresses). For example, this can comprise vertical acceleration data or pitch angle data.

In various cases, for each type of pavement distress, the training sensor dataset can include training sets captured for vehicles travelling at different speeds, or different weights of vehicles or vehicles travelling in different weather conditions. In particular, as previously stated, the sensor dataset responses can vary based on the vehicle speed, weight, or otherwise, weather related conditions (e.g., rainy or dry weather).

At 904c, in some cases, each training dataset can be pre-processed (e.g., applying a noise reduction filter).

At 906c, the training datasets can be labeled, as for example based on the type of sensed pavement distress as well as the severity of the pavement distress. For example, a user operating a user terminal may manually label each training sensor dataset with type and severity information.

At 908c, the labeled sensor data sets are input into an untrained machine learning model (e.g., a CNN model), to generate a trained machine learning model at 910c.

As previously stated, in some embodiments, the method 900c may be iterated to generate different trained machine learning models for different types of sensor data (e.g., motion or orientation data). Otherwise, a single trained machine learning model may be generated for all types of sensor data.

Referring now to FIG. 11, there is shown an example embodiment of a method 1100a for determining pavement condition outputs for a pavement segment based on one or more of image and sensor data. Method 1100 can correspond to act 606 of method 600. In various cases, method 1100 can be executed by one or more of the edge device processor 302a and/or server processor 302b.

At 1102, pavement distress data generated for a pavement segment from image data is received (e.g., a JSON file generated by method 700a of FIG. 7A). For example, this can include data stored in the edge device or server memories 302a, 302b.

At 1104, pavement distress data generated for a pavement segment from sensor data (e.g., motion and/or orientation data) is also received (e.g., a JSON file generated by method 900a of FIG. 9A, or method 900b of FIG. 9B). For example, this can also include data stored in the edge device or server memories 302a, 302b.

At 1106, an aggregated pavement distress dataset in respect of a pavement segment is generated by combining the pavement distress data at 1102, and the pavement distress data at 1104. In other words, the aggregated pavement distress dataset can include all pavement distresses identified from the image data and the sensor data set, as well as all related attributes identified for these pavement distresses.

At 1108, based on the aggregated pavement distress dataset, one or more pavement condition outputs are generated. For example, this can include determining a PCI value. For example, in accordance with method 100c,e by parsing the aggregated pavement distress dataset—the density of each distress type at each severity level can be determined (112c). Further, based on the density information, one or more deduct values may be calculated (114c), as well as a maximum corrected deduct value (mCDV). Based on the determined mCDV, a PCI value can be generated for the pavement segment.

In various cases, to enable determination of PCI values, the various deduct value graphs, as well as the various formulaic relationships required to determining the mCDV may be pre-stored in the edge device or server memory 304a, 304b.

In some embodiments, where multiple images are captures of the same pavement segment, the PCI for the pavement segment may be determined by averaging the PCI determination resulting from each one of these images (e.g., PCI from a given image and the corresponding sensor data).

Referring now to FIG. 12, which shows a process flow for an example embodiment of a method 1200 for assessing pavement condition in accordance with other standards. In particular, method 1200 allows for determining pavement condition according to a regional MTO-SPO24 standard, developed by the Ministry of Transportation of Ontario.

At 1202, the location and attributes of pavement defects on a pavement segment are determined. This can be determined in accordance with method 700a of FIG. 7A as well as methods 900a of FIG. 9A or method 900b of FIG. 9B.

At 1204, the image data captured for a pavement segment may be further analyzed to identify and parse different lane segments. For instance, in accordance with the regional MTO-SPO24 standard, this may involve determining: (i) an edge portion of a pavement segment; (ii) a wheel track portion of pavement segment; and (iii) a center portion of pavement segment. In at least some embodiments, a trained machine learning model (e.g., a lane detection model) is applied to analyze images of the pavement segment with a view to identifying, or boxing, different lane segments.

In some cases, acts 1202 and 1204 may be reversed such that act 1204 is performed before act 1202. In other cases, acts 1202 and 1204 may be performed concurrently, or at least partially, concurrently.

At 1206, each pavement defect identified at act 1202 is associated with a corresponding lane section identified at act 1204. This is based on identifying which lane section the pavement defect is observed in. This information may then be used to calculate a condition index for the pavement section, in accordance with the relevant standard (e.g., MTO-SPO24 standard).

The SPO-24 standard also includes road condition rating (RCR) values which indicate the ride quality. This can be determined using an edge device's accelerometer data.

For example, this can be performed by applying a filter (e.g., a Kalman filter) to read the accelerometer data, and then locating the mean of the filtered data as the RCR value for that pavement segment. In at least one example case, the accelerometer can be operated to recorded data at a frequency of twenty (20) data points per second.

Referring now to FIG. 13A, which shows an example architecture 1300a for a machine learning model that can be used at act 706a in method 700a to process input images of pavement segments and identify locations and classify types of pavement defects in the pavement segment. The architectures 1300a may be stored, for example, on the server memory 304b.

In general, architecture 1300a includes two parts: (i) the first part 1301a involves identifying the locations of the pavement defects in an input image and marking the identified locations (1304a-1308a); and (ii) once the locations are identified, the second part 1303a involves classifying the types of defects within the marked locations (1310a-1312a). In some cases, the locations of the pavement defects are marked by placing bounding boxes around the identified defect. In such cases, the second part 1303a involves classifying the defect marked by the bounding box into a defect classification category. In some other cases, the locations of the pavement defects may be marked by a star, a triangle, any other shape, or any other indicia understood within the architecture 1300a.

In more detail, as shown, at 1302a, an input image is received. For example, this may be an image of a pavement segment captured by an edge device 208 (e.g., image 1300e of FIG. 13E).

At 1304a, one or more trained feature extraction layers 1304a may be applied to the input image. The trained feature extraction layers extract features (e.g., image features) which may indicate the presence or absence of a pavement defect. In particular, the feature extraction layers can generate a feature map indicating where certain features are located within the input image.

At 1306a, the input image may also be analyzed to generate a set of candidate regions, also referred to herein as anchor regions. Anchors are fixed bounding regions that are placed throughout the input image with different sizes and ratios that are used for reference when initially predicting object locations (i.e., locations of pavement distresses in the input image). In some cases, the candidate regions may comprise bounding boxes, or any other suitable bounding shape. For example, as shown in FIG. 13E, in respect of one or more pixels/point 1302e₁, 1302e₂ in the image, a first bounding box 1304e₁, 1304e₂ of a first size (e.g. size ‘A’) may be defined, as well as a second bounding box 1306e₁, 1306e₂ of a different size (e.g. size ‘B’) may be defined. This may be repeated for a plurality of selected pixels in the image. As provided herein, each bounding box may define a potential region in the image where a pavement defect 1308e is located.

In one example, with respect of each image point 1302e₁, 1302e₂, five different sizes of anchors or bounding boxes are used: 64 pixel, 128 pixel, 256 pixel, 512 pixel, and 1024 pixel (i.e., expressing the area of the anchor bounding box in pixels). Accordingly, a variety of sizes of bounding boxes are defined around each image pixel. This, in turn, can enable identifying smaller or larger pavement defects with more accuracy. For example, a larger pavement defect may be identified inside a larger size bounding box, while a smaller pavement defect may be identified with a smaller size bounding box.

In some cases, for each image point 1302e₁, 1302e₂—and for each size of bounding box—a bounding box can be defined when the image 1300e is in portrait mode, and when the image is also in landscape mode. That is, if there are five different sizes of bounding boxes, a total of ten bounding boxes are defined around each image point to cover both the portrait and landscape orientations. This may also be done to enhance the accuracy of locating pavement defects within the image.

To this end, bounding boxes can be defined around any number of image pixels. A larger number of image pixels selected as seeding points for bounding boxes may allow for enhanced accuracy in capturing the particular location of pavement defects. As well, a large diversity of bounding boxes defined around each selected image pixel may also enhance the accuracy of identifying pavement defect location.

In the various embodiments illustrated herein, as the model uses a convolutional feature map (1304a) of size convulution_width×convulution_height×convulution_depth—a set of anchors are generated for each of the points in convulution_width×convulution_height×convulution_depth. In various cases, even though anchors are defined based on the convolutional feature map, the final anchors reference the original image.

Next, the output of the feature extraction (1304a) and generated anchors (1306a) are processed by a regional proposal network (RPN) layer 1308a. The RPN layer 1308a determines which anchor regions overlap with pavement defects. That is, the RPN layer 1308a classifies the anchor regions (1306a) with positive or negative labels. A positive label is assigned to anchor regions that overlap with, or surround, a pavement defect, as detected by the feature map (1304e₁ and 1306e₁ in FIG. 13E). Further, a negative label is assigned to anchor regions that do not overlap with a pavement defect as also identified by the feature map (1304e₂ and 1306e₂ in FIG. 13E). The output of the RPN layer 1308a is then a set of “proposals”, wherein each proposal comprises a positive label anchor bounding region. In some cases, the RPN layer 1308a may also adjust the size of the anchor region to provide a more accurate fit around the detected pavement defect.

At 1310a and 1312a, the pavement defects identified in the proposals—as well as the extracted features from 1304a—are used to predict the type of pavement defect (e.g., the type of pavement crack).

In particular, the ROI (Region of Interest) pooling layer 1310a receives the proposal from the RPN layer 1308a, as well as the feature map from the feature extraction layers 1304a. The purpose of the ROI pooling layer 1310a is to prepare the data for prediction by the regressor 1312a.

More particularly, the ROI pooling takes each region of interest (i.e., each proposal) and identifies a section of the input feature map which corresponds to that ROI, and then converts that feature-map section into a fixed dimension map. In this manner, the feature map for each ROI is standardized by the ROI pooling layer 1310a. This, in turn, prepares the data for prediction (i.e., of the pavement defect) by the regressor 1312a.

Assuming the proposal size is M*N, the fixed feature map size is expressed as pooled_width*pooled_height, the ROI pooling layer 1310a: (i) initially, obtains the ROI by transferring the proposal from the real image space to the feature map space, i.e., 1/16 scale; (ii) divides the ROI by dividing the feature map area that corresponds to the proposal into pooled_width*pooled_height grids, and performing round down if it cannot divide exactly; and (iii) performing max pooling—i.e., run max pooling for each grid.

At 1312a, the output of the ROI pooling 1310a is fed into the regressor 1312a. The objective of the regressor is to analyze each output of the ROI 1310a to predict the output (i.e., the pavement defect type). That is, the regressor functions to analyze the output of the ROI pooling layer 1310a to predict the type of pavement defect in each positive label proposal. In some examples, the regressor is the R-CNN layer, which receives proposal feature maps from ROI pooling layer and runs the detection regression.

At 1314a, the shape of the bounding boxes, around each pavement defect, may be refined in view of the output of the regressor 1312a.

Reference is now briefly made to FIG. 13B, which shows an example architecture 1300b of the RPN layer 1308a, of FIG. 13A. As shown, at 1302b, the RPN layer 1300b receives the feature map from 1304a and the bounding boxes from 1306a. The RPN layer 1308a now operates to map features associated with pavement defects, with specific bounding boxes. In this manner, the RPN layer 1308a can identify the specific bounding boxes which surround pavement defects. In turn, this enables the system to identify the locations of the pavement defects in the image.

To this end, as shown in FIG. 13B, the RPN layer 1308a has two flows: (a) a first flow 1304b is for classifying the anchors (1306a) with positive or negative labels. A positive label is assigned to anchor boxes that overlap with a pavement defect, as detected by the feature map (1304e₁ and 1306e₁ in FIG. 13E). Further, a negative label is assigned to anchor boxes that do not overlap with a pavement defect as identified by the feature map (1304e₂ and 1306e₂ in FIG. 13E); and (b) a second flow 1306b for determining the offset of bounding box regression. In FIG. 13B, the second flow 1312b is shown as being performed in parallel to the first flow 1304b. In other cases, however, the second flow 1312b may be performed after the first flow 1306a. For example, once the positive anchor regions are identified (first flow 1304b), the second flow 1306b may then perform a bounding box regression for only those anchor regions. The outputs from each flow 1304b and 1306b are then combined into a proposal layer 1316b, which generates and filters suitable proposals (i.e., regions that identify the locations of pavement defects).

With respect to the first flow 1404b, this involves applying a convolution layer 1307b, followed by reshaping bounding boxes around identified pavement defect features at 1308b, 1312b and applying a SoftMax function in between at 1310b. With respect to the second flow 1314b, this involve applying one or more convolution layers. The output 1316b corresponds to one or more “proposals”, where each proposal corresponds to a bounding box around a pavement defect area.

In more detail, the upper flow 1304b sets up a dense number of candidate anchors on the scale of the original image, and then uses the base network to classify which anchors are positive (i.e., covering the ground truth) and which are negative anchors (i.e., outside the ground truth). Eventually, the method turns out to solving a binary classification problem.

Referring now to FIG. 13C, which shows an example architecture 1300c for the regressor 1312a. As shown, the regressor 1312a has two fully-connected layers (i.e., of size 4096) 1302c, 1306c with corresponding rectified linear unit (ReLU) activation 1304c, 1308c. This is then followed by one convolution layer 1310c with 4N units (N is the number of classes) of [center_x, center_y, width, height]. Each of the single convolution layers 1302c, 1306c using 1×1 convolution filters.

Referring to FIG. 13D, which shows different possible example architectures 1300d of the feature extraction layers 1304a, which comprise a convolutional neural network (CNN). In particular, the architecture 1300d is a ResNet architecture (i.e. a Residual neural network architecture), and for convolution layers 2_x to 5_x, FIG. 13D shows various parameters for an 18-layer, 24-layer, 50-layer, 101-layer and 152-layer architecture configuration. The parameters shown for these layer types are the convolution layer size (i.e., width×height×channel). For example, under conv2_x for 18-layer: “3×3, 64” denotes a convolution layer with a width of 3 pixels×a height of 3 pixels and 64 channels. The “2” outside the matrix (i.e., “x2”) denotes the number of layers (i.e., repeating the same structure in the matrix twice). Further, the term FLOPS references Floating Point Operations Per Second. In one example, the CNN used is a ResNet-50 model having the 50-layer architecture and output sizes shown in FIG. 13D. To this end, the feature extraction layers 1304a extract features that enable the system to identify properties of the image that facilitates the identification of the location of defects. In some other cases, other types of CNN models, such as, for example, GoogleNet, PolyNet, LeNet, ResNeXt-50, among others, may be used at stage 1304a.

In at least one embodiment, the model in method 1300a is trained using an input image data set as previously described. The images in the dataset can be generated using front view images taken by a camera mounted on the vehicle's windshield. In one example, the dataset has 57 classes and each class having around 200 images. Within each image, the pavement defects are labelled or annotated with bounding boxes, and the pavement defect in the bounding box is pre-classified. In this example, the model 1300a is trained with a 0.01 learning rate, 8 images per batch, and 5000 epochs.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. A method for assessing a condition of a pavement segment, the method comprising:
analyzing, using a trained machine learning model, an image of the pavement segment;
generating, by the machine learning model, an output comprising one or more identified first pavement distresses in the pavement segment;
analyzing accelerometer data associated with the pavement segment;
based on the analyzing, determining one or more identified second pavement distresses in the pavement segment; and
based on the one or more identified first and second pavement defects, determining pavement condition data associated with the pavement segment.

2. The method of claim 1, wherein the image and accelerometer data are captured by a camera and accelerometer of an edge device.

3. The method of claim 2, wherein the edge device is mounted to a vehicle.

4. The method of claim 1, further comprising analyzing the pavement condition data to determine one or more pavement condition outputs, associated with the pavement segment.

5. The method of claim 4, wherein the pavement condition output corresponds to a pavement condition index (PCI) value.

6. The method of claim 1, wherein analyzing the image using the trained machine learning model comprises:
applying feature extraction layers to the image to generate a feature map;
generating a set of anchor boxes in the image to generate a plurality of anchor boxes;
inputting the plurality of anchor boxes and the feature map into a regional proposal network (RPN) layer to identify the locations of the pavement defects in the image.

7. The method of claim 6, wherein the feature extraction layer comprises a ResNet-50 model.

8. The method of claim 6, further comprising the trained machine learning model classifying the pavement defect types.

9. The method of claim 8, wherein classifying the pavement defect types is generated by feeding the feature extraction map and proposal generated by the RPN layer, into an ROI (region of interest) pooling layer and a regressor.

10. The method of claim 1, wherein prior to applying the trained machine learning, applying a tilt correction to the input image.

11. A system for assessing a condition of a pavement segment, the system comprising:
an edge device comprising an image sensor for capturing an image of the pavement segment, and at least one of a motion and orientation sensor for recording respective motion and orientation data associated with the pavement segment; and
a server comprising a processor configured to:
receive, from the edge device, the image and at least one of motion and orientation data;
analyze, using a trained machine learning model, the image of the pavement segment;
generate, by the machine learning model, an output comprising one or more identified first pavement distresses in the pavement segment;
analyze at least one of the motion and orientation data associated with the pavement segment;
based on the analyzing, determine one or more identified second pavement distresses in the pavement segment; and
based on the one or more identified first and second pavement defects, determine a pavement condition output associated with the pavement segment.

12. The system of claim 11, wherein the edge device is mounted to a vehicle.

13. The system of claim 11, wherein the motion data comprises accelerometer data.

14. The system of claim 11, wherein the processor is further configured to analyze the pavement condition data to determine one or more pavement condition outputs, associated with the pavement segment.

15. The system of claim 14, wherein the pavement condition output corresponds to a pavement condition index (PCI) value.

16. The system of claim 11, wherein analyzing the image using the trained machine learning model comprises the processor being further configured to:
apply feature extraction layers to the image to generate a feature map;
generate a set of anchor boxes in the image to generate a plurality of anchor boxes;
input the plurality of anchor boxes and the feature map into a regional proposal network (RPN) layer to identify the locations of the pavement defects in the image.

17. The system of claim 16, wherein the feature extraction layer comprises a ResNet-50 model.

18. The system of claim 16, further comprising the trained machine learning model classifying the pavement defect types.

19. The system of claim 16, wherein classifying the pavement defect types is generated by feeding the feature extraction map and proposal generated by the RPN layer, into an ROI (region of interest) pooling layer and a regressor.

20. The system of claim 11, wherein prior to applying the trained machine learning, applying a tilt correction to the input image.
